SEQUENCE LISTING (40 sequences)

SEQ ID NO: 1 - protein, 7 aa, Artificial Sequence
  synthetic fiber protein sequence for generating antibodies reactive to lipogenic adenovirus
FNPVYPY

SEQ ID NO: 2 - protein, 12 aa, Artificial Sequence
  synthetic fiber protein sequence for generating antibodies reactive to lipogenic adenovirus
NIPFLTPPFVSS

SEQ ID NO: 3 - protein, 15 aa, Artificial Sequence
  synthetic fiber protein sequence for generating antibodies reactive to lipogenic adenovirus
FPPGVLSLKLADPIA

SEQ ID NO: 4 - protein, 17 aa, Artificial Sequence
  synthetic fiber protein sequence for generating antibodies reactive to lipogenic adenovirus
ANGNVSLKVGGGLTVEQ

SEQ ID NO: 5 - protein, 14 aa, Artificial Sequence
  synthetic fiber protein sequence for generating antibodies reactive to lipogenic adenovirus
SGKLSVDTKAPLQV

SEQ ID NO: 6 - protein, 9 aa, Artificial Sequence
  synthetic fiber protein sequence for generating antibodies reactive to lipogenic adenovirus
AGHGLAVVT

SEQ ID NO: 7 - protein, 13 aa, Artificial Sequence
  synthetic fiber protein sequence for generating antibodies reactive to lipogenic adenovirus
SLPSLVGTLVVLT

SEQ ID NO: 8 - protein, 7 aa, Artificial Sequence
  synthetic fiber protein sequence for generating antibodies reactive to lipogenic adenovirus
PSPNCKV

SEQ ID NO: 9 - protein, 32 aa, Artificial Sequence
  synthetic fiber protein sequence for generating antibodies reactive to lipogenic adenovirus
SKLTLALTKCGSQILATVSLLVVTGKYAIISD

SEQ ID NO: 10 - protein, 20 aa, Artificial Sequence
  synthetic fiber protein sequence for generating antibodies reactive to lipogenic adenovirus
NPKQFSIKLLFNDKGVLLSD

SEQ ID NO: 11 - protein, 7 aa, Artificial Sequence
  synthetic fiber protein sequence for generating antibodies reactive to lipogenic adenovirus
YKEAVGF

SEQ ID NO: 12 - protein, 14 aa, Artificial Sequence
  synthetic fiber protein sequence for generating antibodies reactive to lipogenic adenovirus
LGGEVYQPGFIVVK

SEQ ID NO: 13 - protein, 7 aa, Artificial Sequence
  synthetic fiber protein sequence for generating antibodies reactive to lipogenic adenovirus
ANCAYSI

SEQ ID NO: 14 - protein, 12 aa, Artificial Sequence
  synthetic fiber protein sequence for generating antibodies reactive to lipogenic adenovirus
WGKVYKDPIPYD

SEQ ID NO: 15 - protein, 10 aa, Artificial Sequence
  synthetic fiber protein sequence for generating antibodies reactive to lipogenic adenovirus
VETARDSKLT

SEQ ID NO: 16 - protein, 14 aa, Artificial Sequence
  synthetic fiber protein sequence for generating antibodies reactive to lipogenic adenovirus
LGGEVYQPGFIVVK

SEQ ID NO: 17 - protein, 12 aa, Artificial Sequence
  synthetic fiber protein sequence for generating antibodies reactive to lipogenic adenovirus
WGKVYKDPIPYD

SEQ ID NO: 18 - protein, 8 aa, Artificial Sequence
  synthetic fiber protein sequence for generating antibodies reactive to lipogenic adenovirus
GTGSSAHG

SEQ ID NO: 19 - DNA, 20 bp, Artificial Sequence
  synthetic Ad-36 hexon protein PCR amplification forward primer
ggtggacaaccacccactac

SEQ ID NO: 20 - DNA, 20 bp, Artificial Sequence
  synthetic Ad-36 hexon protein PCR amplification forward primer
tggacaaccacccactacaa

SEQ ID NO: 21 - DNA, 20 bp, Artificial Sequence
  synthetic Ad-36 hexon protein PCR amplification reverse primer
cagcggagcttgtatcttcc

SEQ ID NO: 22 - DNA, 23 bp, Artificial Sequence
  synthetic Ad-36 fiber protein nested PCR outer forward primer
gtctggaaaactgagtgtggata

SEQ ID NO: 23 - DNA, 24 bp, Artificial Sequence
  synthetic Ad-36 fiber protein nested PCR outer reverse primer
atccaaaatcaaatgtaatagagt

SEQ ID NO: 24 - DNA, 22 bp, Artificial Sequence
  synthetic Ad-36 fiber protein nested PCR inner forward primer
ttaactggaaaaggaataggta

SEQ ID NO: 25 - DNA, 24 bp, Artificial Sequence
  synthetic Ad-36 fiber protein nested PCR inner reverse primer
ggtgttgttggttggcttaggata

SEQ ID NO: 26 - DNA, 25 bp, Artificial Sequence
  synthetic probe to detect Ad-36 fiber coat protein
agttgaaacagcaagagactcaaag

SEQ ID NO: 27 - DNA, 25 bp, Artificial Sequence
  synthetic probe to detect Ad-36 fiber coat protein
ggtactggatcaagtgcacatggag

SEQ ID NO: 28 - DNA, 28 bp, Artificial Sequence
  synthetic probe to detect Ad-36 fiber coat protein
ttgaaacagcaagagactcaaagctaac

SEQ ID NO: 29 - DNA, 33067 bp, Adenovirus type 36
  modified_base (1)...(33067): n = g, a, c or t
taacgaagaggctgtgaatttaatatttccagaatctatgattcttcaggctgacatagccagtgaagccatagttactcctctacatactcccactctgcctcccatacctgaattggaggaggatgaagaaatagacctccggtgctacgaggaaggttttcctcccagcgattcagaggacgaacagggtgagcagcagatggctctaatctctgatttagcttgtgtgattgtggaggaacaagttgtgattgaaaaatctaccgagccagtacaaggctgtaggaactgccagtatcaccgggataagtccggagacccgaacgcttcctgcgctctgtgttacatgaaatctactttcagctttatttacagtaagtggagtgaatgtgagagaggctgagtgcttaacacataactgtaatgcttgaacagctgtgctaagtgtggtttatttttgttactaggtccggtgtcagaggatgagtcatcaccctcagaagaagaccacccgtctccccctgagctgtcaggcgaaacgcccctgcaagtgcacagacccaccccagtcagagccagtggcgagaggcgagcagctgtagaaaaaattgaggacttgttacatgacatgggtggggatgaacctttggacctgagcttgaaacgccccaggaactaggcgcagctgcgcttagtcatgtgtaaataaagttgtacaataaaagtatatgtgacgcatgcaaggtgtggtttatgactcatgggcggggcttagtcctatataagtggcaacacctgggcacttgggcacagaccttcagggagttcctgatggatgtgtggactatccttgcagactttagcaagacacgccggcttgtagaggatagttcagacgggtgctccgggttctggagacactggtttggaactcctctatctcgcctggtgtatacagttaagaaggattataaagaggaatttgaaaatctttttgctgactgctctggtctgctagattctctgaatcttggccaccagtcccttttccaggaaagggtactccacagccttgatttttccagcccagggcgcactacagccggggttgcttttgtggtttttctggttgacaaatggagccaggacacccaactgagcaggggctacatcctggacttcgcagccatgcacctgtggagggcctggatcaggcagcggggacagagaatcttgaactactggcttctacagccagcagctccgggtcttcttcgtctacacagacaaacatccatgttggaggaagaaatgaggcaggccatggacgagaacccgaggagcggcctggaccctccgtcggaagaggagctggattgaatcaggtatccagcctgtacccagagcttagcaaggtgctgacatccatggccaggggagtgaagagggagaggagcgatgggggtaataccgggatgatgaccgagctgactgccagtctgatgaatcggaagcgcccagagcgccttacctggtacgagctacagcaggagtgcagggatgagataggcctgatgcaggataaatatggcctggagcagataaaaacccattggttgaacccagatgaggattgggaggaggctattaagaagtatgccaagatagccctgcgcccagattgcaagtacatagtgaccaagaccgtgaatatcagacatgcctgctacatctcggggaacggggcagaggtggtcatcgataccctggacaaggccgccttcaggtgttgcatgatgggaatgagagcaggagtgatgaatatgaattccatgatcttcatgaacattaagttcaatggagagaagtttaatggggtgctgttcatggccaacagccacatgaccctgcatggctgcagcttcttcggtttcaacaacatgtgcgccgaggtctggggagctgctaagatcaggggatgtaagttttatggctgctggatgggcgtggtcggaagacccaagagcgagatgtctgtgaagcagtgtgtgtttgagaaatgctacctgggagtctctaccgagggcaatgctagagtgagacactgctcttccatggagacgggctgcttctgcctggtgaagggcacggcctctctgaagcataatatggtgaagggctgcacggatgagcgcatgtacaacatgctgacctgcgattcgggggtctgccatatcctgaagaacatccatgtgacctcccaccccagaaagaagtggccagtgtttgagaataacctgctgatcaagtgccatatgcacctgggtgccagaaggggcaccttccagccgtaccagtgcaactttagccagaccaagctgctgttggagaacgatgccttctccagggtgaacctgaacggcatctttgacatggatgtctcggtgtacaagatcctgagatacgatgagaccaagtccagggtgcgcgcttgcgagtgcgggggcagacacaccaggatgcaaccagtggccctggatgtgacagaggagctgagaccagaccacctggtgatggcctgtaccgggaccgagttcagctccagtggggaggacacagattagaggtaggttttgagtagtgggcgtggctaaggtgagtataaaggcggtgtcttacgagggtctttttgcttttctgcagacatcatgaacgggaccggcggggccttcgaaggggggctttttagcccttatttgacaacccgcctgccgggatgggccggagttcgtcagaatgtgatgggatctacggtggatgggcgcccagtgcttccagcaaattcctcgaccatgacctacgcgaccgtggggagctcgtcgctcgacagcaccgccgcagccgcggcagccgcagccgccatgacagcgacgagactggcctcgagctacatgcccagcagcagcagtagcccytctgtgcccagttccatcatcgccgaggagaaactgctggccctgctggcagagctggaagccctgagccgccagctggccgccctgacccagcaggtgtccgagctccgcgagcaacagcagcagcaaaataaatgattcaataaacacagattctgattcaaacagcaaagcatctttattatttattttttcgcgcgcggtaggccctggtccacctctcccgatcattgagagtgcggtggattttttccaggacccggtagaggtgggattggatgttgaggtacatgggcatgagcccgtcccgggggtggaggtagcaccactgcatggcctcgtgctctggggtcgtgttgtagatgatccagtcatagcaggggcgctgggcgtggtgctggatgatgtccttaaggaggagactgatggccacggggagccccttggtgtaggtgttggcgaagcggttgagctgggagggatgcatgcggggggagatgatgtgcagtttggcctggatcttgaggttggcaatgttgccgcccagatcccgcctggggttcatgttgtgcaggaccaccaggacggtgtagcccgtgcacttggggaacttatcatgcaacttggaagggaatgcgtggaagaatttggagacgcccttgtgcccgcccaggttttccatgcactcatccatgatgatggcgatgggcccgtgggctgcggctttggcaaagacgtttctggggtcagagacatcgtaattatgctcctgggtgagatcatcataagacattttaatgaatttggggcggagggtgccagattgggggacgatagttccctcgggccccggggcaaagttcccctcacagatctgcatctcccaggctttcatctcggagggggggatcatgtccacctgcggggcgatgaaaaaaacggtttccggggcgggggtgatgagctgcgaggagagcaggtttctcaacagctgggacttgccgcacccggtcgggccgtagatgaccccgatgacgggttgcaggtggtagttcaaggacatgcagctgccgtcgtcccggaggaggggggccacctcgttgagcatgtctctgacttggaggttttcccggacgagctcgccgaggaggcggtccccgcccagcgagaggagctcttgcagggaagcaaagtttttcaggggcttgagtccgtcggccatgggcatcttggcgagggtctgcgagaggagctccaggcggtcccagagctcggtgacgtgctctacggcatctcgatccagcagacttcctcgtttcgggggttgggacgactgcgactgtagggcacgagacgatgggcgtccagcgcggccagcgtcatgtccttccagggtctcagggtccgcgtgagtgtggtctccgtcacggtgaaggggtgggccccgggctgggcgcttgcaagggtgcgcttgagactcatcctgctggtgctgaaacgggcacggtcttcgccctgcgcgtcggcgagatagcagttgaccatgagctcgtagttgagggcctcggcggcgtggcccttggcgcggagcttgcccttggaagagcgcccgcaggcgggacagagaagggattgcagggcgtagagcttgggtgcgagaaaaacggactcgggggcgaaggcgtccgctccgcagtgggcgcagacggtctcgcactcgacgagccaggtgagctcgggctgctcggggtcaaaaaccagttttcccccgttctttttgatgcgcttcttacctcgcgtctccatgagtctgtgtccgcgctcggtgacaaacaggctgtcggtgtccccgtagacggacttgatgggcctgtcctgcagggacgtcccgcggtcctcctcgtagagaaactcggaccactctgagacaaaggcgcgcgtccacgccaagacaaaggaggccacgtgcgaggggtagcggtcgttgtccaccagggggtccaccttttccaccgtgtgcagacacatgtccccctcctccgcatccaagaaggtgattggcttgtaggtgtaggccacgtgaccgggggtccccgacgggggggtataaaagggggcgggtctgtgctcgtcctcactctcttccgcgtcgctgtccacgagcgccagctgttggggtaggtattccctctctagagcgggcatgacctcggcactcaggttgtcagtttctagaaacgaggaggatttgatgttggcctgccctgccgcgatgctttttaggagactttcatccatctggtcagaaaagacaatttttttattgtcaagcttggtggcaaaggagccatagagggcgttggatagaagcttggcgatggatctcatggtctgatttttgtcacggtcggcgcgctccttggccgcgatgtttagctggacatactcgcgcgcgacgcacttccattcggggaagacggtggtgcgctcgtcgggcacgatcctgacgcgccagccgcggttatgcagggtgaccaggtccacgctggtggccacctcgccgcgcaggggctcgttggtccagcagagtctgccgcccttgcgcgagcagaacgggggcagcacatcaagcagatgctcgtcaggggggtccgcatcgatggtgaagatgcccggacagagttccttgtcaaaataatcgatttttgaggatgcatcatccaaggccatctgccactcgcgggcggccagcgctcgctcgtaggggttgaggggcggaccccagggcatgggatgcgtgagggcggaggcgtacatgccgcagatgtcatacacatagatgggctccgagaggatgccgatgtaggtgggataacagcgccccccgcggatgctggcgcgcacgtagtcatacaactcgtgcgagggggccaagaaggcggggccgagattggtgcgctggggctgctcggcgcggaagacgatctggcgaaagatggcgtgcgagtttgaggagatggtgggccgttggaagatgttaaagtgggcgtgaggcaggcggaccgagtcgcggatgaagtgcgcgtaggagtcttgcagcttggcgacgagctcggcggtgacgaggacgtccatggcacagtagtccagcgtttcgcggatgatgtcataacccgcctctcctttcttctcccacagctcgcggttgagggcgtactcctcgtcatccttccagtactcccggagcgggaatcctcgatcgtccgcacggtaagagcccagcatgtagaaatggttcacggccttgtagggacagcagcccttctccacggggagggcgtaagcttgagcggccttgcggagcgaggtgtgcgtcagggcgaaggtgtctctgaccatgactttcaagaactggtacttgaaatccgagtcgtcgcagccgccgtgctcccagagctcgaaatcggtgcgcttcttcgagagggggttaggcagagcgaaagtgacgtcattgaagagaatcttgcctgcccgcggcatgaaattgcgggtgatgcggaaagggcccgggacggaggctcggttgttgatgacctgggcggcgagcacgatctcgtcgaagccgttgatgttgtgcccgacgatgtagagttccatgaatcgcgggcggcctttgatgtgcggcagctttttgagctcctcgtaggtgaggtcctcggggcattgcagtccgtgctgctcgagcgcccactcctggagatgtgggttggcttgcatgaaggaagcccagagctcgcgggccatgagggtctggagctcgtcgcgaaagagacggaactgctggcccacggccatcttttcgggtgtgacgcagtagaaggtgagggggtcccgctcccagcgatcccagcgtaagcgcacggcgagatcgcgagcgagggcgaccagctcggggtccccggagaatttcatgaccagcatgaaggggacgagctgcttgccgaaggaccccatccaggtgtaggtttctacatcgtaggtgacaaagagccgctccgtgcgaggatgagagccgattgggaagaactggatttcctgccaccagttggacgagtggctgttgatgtgatgaaagtagaaatcccgccggcgaaccgagcactcgtgctgatgcttgtaaaagcgtccgcagtactcgcagcgctgcacgggctgtacctcatccacgagatacacagcgcgtcccttgaggaggaacttcaggagtggcggccctggctggtggttttcatgttcgcctgcgtgggactcaccctggggctcctcgaggacggagaggctgacgagcccgcgcgggagccaggtccagatctcggcgcggcgggggcggagagcgaagacgagggcgcgcagttgggagctgtccatggtgtcgcggagatccaggtccgggggcagggttctgaggttgacctcgtagaagcgggtgagggcgtgcttgagatgcagatggtacttgatttctacgggtgagttggtggccgtgtccacgcattgcatgagcccgtagctgcgcggggccacgaccgtgccgcggtgcgcttttagaagcggtgtcgcggacgcgctcccggcggcagcggcggttccggccccgcgggcaggggcggcagaggcacgtcggcgtggcgctcgggcaggtcccggtgctgcgccctgagagcgctggcgtgcgcgacgacgcggcggttgacatcctggatctgccgcctctgcgtgaagaccacgggccccgtgactttgaacctgaaagacagttcaacagaatcaatctcggcgtcattgacggcggcctgacgcaggatctcttgcacgtcgcccgagttgtcctggtaggcgatctcggacatgaactgctcgatctcctcctcctggagatcgccgcggcccgcgcgctccacggtggcggcgaggtcattcgagatgcgacccattagctgcgagaaggcgcccaggccgctctcgttccagacgcggctgtagaccacgtccccgtcggcgtcgcgcgcgcgcatgaccacctgcgcgaggttgagctccacgtgccgcgtgaagacggcgtagttgcgcaggcgctggaagaggtagttgagggtggtggcgatgtgctcggtgacgaagaagtacatgatccagcggcgcaggggcatctcgctgatgtcgccgatggcctccagcctttccatggcctcgtagaaatccacggcgaagttgaaaaactgggcgttgcgggccgagaccgtgagctcgtcttccaggagccggatgagctcggcgatggtggcgcgcacctcgcgctcgaaatccccggggacctcctcctcttcctcttcttccatgacgacctcttcttctatttcttcctctgggggcggtggtggtggcggggcccgacgacgacggcgacgcaccgggagacggtcgacgaagcgctcgatcatctccccgcggcggcgacgcatggtttcggtgacggcgcgaccccgttcgcgaggacgcagcgtgaagacgccgccggtcatctcccggtaatggggcgggtccccgttgggcagcgatagggcgctgacgatgcatcttatcaattgcggtgtaggggacgtgagcgcgtcgagatcgaccggatcggagaatctttcgaggaaagcgtctagccaatcgcagtcgcaaggtaagctcaaacacgtagcagccctgtggacgctgttagaattgcggttgctgatgatgtaattgaagtaggcgtttttaaggcggcggatggtggcgaggaggaccaggtccttgggtcccgcttgctggatgcggagccgctcggccatgccccaggcctggccctgacaccggctcaggttcttgtagtagtcatgcatgagcctttcaatgtcatcactggcggaggcggagtcttccatgcgggtgaccccgacgcccctgagcggctgcacgagcgccaggtcggcgacgacgcgctcggcgaggatggcctgttgcacgcgggtgagggtgtcctggaagtcatccatgtcgacgaagcggtggtaggccccggtgttgatggtgtaggtgcagttggccatgagcgaccagttgacggtctgcaggccgggttgcacgacctcggagtacctgagccgcgagaaggcgcgcgagtcgaagacgtagtcgttgcaggtgcgcacgaggtactggtagccgactaggaagtgcggcggcggctggcggtagagcggccagcgctgggtggccggcgcgcccggggccaggtcctcgagcatgaggcggtggtagccgtagaggtagcgggacatccaggtgatgccggcggcggtggtggaggcgcgcgggaactcgcggacgcggttccagatgttgcgcagcggcaggaaataatccatggtcggcacggtctggccggtgagacgcgcgcagtcattgacgctctagaggcaaaaacgaaagcggttgagcgggctcttcctccgtagcctggcggaacgcaaacgggttaggccgcgtgtgtaccccggttcgagtcccctcgaatcaggctggagccgcgactaacgtggtattggcactcccgtctcgacccgagcccgatagccgccaggatacggcggagagccctttttgccgaccgagtggggtcgctagacttgaaagcggccgaaaaccccgccgggtagtggctcgcgcccgtagtctggagaagcatcgccagggttgagtcgcggcagaacccggttcgcggacggccgcggcgagcgggacttggtcaccccgccgatttaaagacccacagccagccgacttctccagttacgggagcgagcccccttttttctttttgccagatgcatcccgtcctgcgccaaatgcgtcccacccccccggcgaccaccgcgaccgcggccgtaacaggcgccggcgctagccagccacagacagagatggacttggaagagggcgaagggctggcgagactgggggcgccgtccccggagcgacacccccgcgtgcagctgcagaaggacgtgcgcccggcgtacgtgcctgcgcagaacctgttcagggaccgcagcggggaggagcccgaggagatgcgcgactgccggtttcgggcgggcagggagctgcgcgagggcctggaccgccagcgcgtgctgcgcgacgaggatttcgagccgaacgagcagacggggatcagccccgcgcgcgcgcacgtggcggcggccaacctggtgacggcctacgagcagacggtgaagcaggagcgcaacttccaaaagagtttcaacaaccacgtgcgcacgctgatagcgcgcgaggaggtggccctgggcctgatgcacctgtgggacctggcggaggccatcgtgcagaacccggacagcaagcctctgacggcgcagctgttcctggtggtgcagcacagcagggacaacgaggcgttcagggaggcgctgctgaacatcgccgagcccgagggtcgctggctgctggagctgatcaacatcttgcagagcatcgtagtgcaggagcgcagcctgagcctggccgagaaggtggcggcgatcaactactcggtgctgagcctgggcaagttttacgcgcgcaagatttacaagacgccgtacgtgcccatagacaaggaggtgaagatagacagcttttacatgcgcatggcgctcaaggtgctgacgctgagcgacgacctgggcgtgtatcgcaacgaccgcatccacaaggccgtgagcacgagccggcggcgcgagctgagcgaccgcgagctgatgctgagcctgcgccgggcgctggtagggggcgccgccggcggcgaggagtcctacttcgacatgggggcggacctgcattggcagccgagccggcgcgccttggaggccgcctacggtccagaggacttggatgaggatgaggaagaggaggaggatgcacccgttgcggggtactgacgcctccgtgatgtgtttttagatgtcccagcaagccccggaccccgccataagggcggcgctgcaaagccagccgtccggtctagcatcggacgactgggaggccgcgatgcaacgcatcatggccctgacgacccgcaaccccgagtcctttagacaacagccgcaggccaacagactctcggccattctggaggcggtggtcccctctcggaccaaccccacgcacgagaaggtgctggcgatcgtgaacgcgctggcggagaacaaggccatccgtcccgacgaggccgggctggtgtacaacgccctgctggagcgcgtgggccgctacaacagcacgaacgtgcagtccaacctggaccggctggtgacggacgtgcgcgaggccgtggcgcagcgcgagcggttcaagaacgagggcctgggctcgctggtggcgctgaacgccttcctggcgacgcagccggcgaacgtgccgcgagggcaggacgattacaccaactttatcagcgcgctgcggctgatggtgaccgaggttccccagagcgaggtgtaccagtcgggcccggactactttttccagacaagccggcagggcctgcagacggtgaacctgagtcaggctttcaagaacctgcgcgggctgtggggcgtgcaggcgcccgtgggcgaccggtcgacggtgagcagcttgctgacgcccaactcgcggctgctgctgctgctgatcgcgcccttcaccgacagcggcagcgtgaaccgcaactcgtacctgggccacctgctgacgctgtaccgcgaggccataggccaggcgcaggtggacgagcagaccttccaggagatcacgagcgtgagccgcgcgctggggcagaacgacaccgacagtctgagggccaccctgaactttttgctgacaaatagacagcagaagatcccggcgcagtacgcactgtcggccgaggaggaaaggatcctgagatatgtgcagcagagcgtagggctgttcctgatgcaggagggtgccacccccagcgccgcgctggacatgaccgcgcgcaacatggaacctagcatgtacgccgccaaccggccgttcatcaataagctgatggactacctgcaccgcgcggcggccatgaacacggactactttacaaacgccatattgaacccgcactggcttccgccgccggggttctacacgggcgagtacgacatgcccgaccccaacgacgggttcctgtgggacgacgtggacagcgcggtgttctcgccgacctttcaaaagcgccaggaggcgccgccgagcgagggcgcggtggggaggagcccctttcctagcttagggagtttgcatagcttgccgggctcggtgaacagcggcagggtgagccggccgcgcttgctgggcgaggacgaatacctgaacgactcgctgctgcagccgccgcgggtcaagaacgccatggccaataacgggatagagagtctggtggacaaactgaaccgctggaagacctacgctcaggaccatagggagcctgcgcccgcgccgcggcgacagcgccacgaccggcagcggggcctggtgtgggacgacgaggactcggccgacgatagcagcgtgttggacttgggcgggagcggtggggccaacccgttcgcgcatctgcagcccagactggggcggcggatgttttgaatgcaaaataaaactcaccaaggccatagcgtgcgttctcttccttgttagagatgaggcgtgcggtggtgtcttcctctcctcctccctcgtacgagagcgtgatggcgcaggcgaccctggaggttccgtttgtgcctccgcggtatatggctcctacggagggcagaaacagcattcgttactcggagctggctccgcagtacgacaccactcgcgtgtacttggtggacaacaagtcggcggacatcgcttccctgaactaccaaaacgaccacagcaacttcctgaccacggtggtgcagaacaacgatttcacccccgccgaggccagcacgcagacgataaattttgacgagcggtcgcggtggggcggtgatctgaagaccattctgcacaccaacatgcccaatgtgaacgagtacatgttcaccagcaagtttaaggcgcgggtgatggtggctagaaagcatcccaaagatgtagatgccagtgatttaagcaaggatatcttagagtataagtggtttgagtttaccctgcccgagggcaacttttccgagaccatgaccatagacctgatgaacaacgccatcttggaaaactacttgcaagtggggcggcagaatggcgtgctggagagcgatatcggagtcaagtttgacagcaggaatttcagactgggctgggacccggtgaccaagctggtgatgccaggggtctacacctacgaggccttccacccggacgtggtgctactgccgggctgcggggtggacttcaccgagagccgcctgagcaacctcctgggcattcgcaagaagcaaccttttcaagagggcttcagaatcatgtatgaggatctagaagggggtaacatccccgctctcctggataccaaaaaatatctggatagcaagaaggaacttgaggatgctgccaaggaagctgcaaagcaacagggagatggtgctgtcactagaggcgatacccacctcactgtagctcaagaaaaagcagctgaaaaggagctagtgatcgtaccaattgaaaaggatgagagcaacagaagttacaacctgatcaaggacacccatgacaccctgtaccgaagctggtacctgtcctatacctacggggaccccgagaagggggtgcagtcgtggacgctgctcaccaccccggacgtcacctgcggcgcggagcaagtctactggtcgctgccggacctcatgcaagaccccgtcaccttccgctctacccagcaagtcagcaactaccccgtggtcggcgccgagctcatgcccttccgcgccaagagcttttacaacgacctcgccgtctactcccagctcatccgcagctacacctccctcacccacgtcttcaaccgcttccccgacaaccagatcctctgccgcccgcccgcgcccaccatcaccaccgtcagtgaaaacgtgcctgctctcacagatcacgggacgcttccgctgcgcagcagtatccgcggagtccagcgagtgaccgtcactgacgcccgtcgccgcacctgtccctacgtctacaaggccctgggcatagtcgcgccgcgcgtgctctccagtcgcaccttctaaaaaatgtctattctcatctcgcccagcaataacaccggctggggtcttactaggcccagcaccatgtacggaggagccaagaagcgctcccagcagcaccccgtccgcgtccgcggtcacttccgcgctccctggggagcttacaagcgggggcgcactgccaccgccgccgccgtgcgcaccaccgtcgacgacgtcatcgactcggtggtcgccgacgcgcgcaactacacccccgccccctccaccgtggacgcggtcatcgacagcgtggtggccgacgcgcgcgactatgccagacgcaagagccggcggcgacggatcgccaggcgccaccggagcacgcccgccatgcgcgccgcccgggctctgctgcgccgcgccagacgcacgggccgccgggccatgatgcgagccgcgcgccgcgccgccactgcaccccccgcaggcaggactcgcagacgagcggccgccgccgctgccgcggccatctctagcatgaccagacccaggcgcggaaacgtgtactgggtgcgcgactccgtcacgggcgtgcgcgtgcccgtgcgcacccgtcctcctcgtccctgatctaatgcttgtgtcctcccccgcaagcgacgatgtcaaagcgcaaaatcaaggaggagatgctccaggtcgtcgccccggagatttacggacccccggaccagaaaccccgcaaaatcaagcgggttaaaaaaaaggatgaggtggacgagggggcagtagagtttgtgcgcgagttcgctccgcggcggcgcgtaaattggaaggggcgcagggtgcagcgcgtgttgcggcccggcacggcggtggtgttcacgcccggcgagcggtcctcggtcaggagcaagcgtagctatgacgaggtgtacggcgacgacgacatcctggaccaggcggcggagcgggcgggcgagttcgcctacgggaagcggtcgcgcgaagaggagctgatctcgctgccgctggacgaaagcaaccccacgccgagcctgaagcccgtgaccctgcagcaggtgctgccccaggcggtgctgctgccgagccgcggggtcaagcgcgagggcgagagcatgtacccgaccatgcagatcatggtgcccaagcgccggcgcgtggaggacgtgctggacaccgtgaaaatggatgtggagcccgaggtcaaggtgcgccccatcaagcaggtggcgccgggcctgggcgtgcagaccgtggacattcagatccccaccgacatggatgtcgacaaaaaaccctcgaccagcatcgaggtgcagaccgacccctggctcccagcctccaccgctaccgtctccacttttaccgccgccacggctaccgagcctcccaggaggcgaagatggggcgccgccagccggctgatgcccaactacgtgttgcatccttccatcatcccgacgccgggctaccgcggcacccggtactacgccagccgcaggcgcccagccgccaaacgccgccgccgcaytgccacccgccgccgtmtggcccccgcccgcgtgcgccgcgtaaccacgcgccggggccgctcgytcgttctgcccaccgtgcgctaccaccccagcatcctttaatccgtgtgctgtgatactgttgcagagagatggctctcacttgccgcctgcgcatccccgtcccgaattaccgaggaagatcccgccgcaggagaggcatggcaggcagcggcctgaaccgccgccggcggcgggccatgcgcaggcgcctgagtggcgggtttctgcccgcgctcatccccataatcgccgcggccatcggcacgatcccgggcatagcttccgttgcgctgcaggcgtcgcagcgccgttgatgtgcgaataaagcctctttagactctgacacacctggtcctgtatatttttagaatggaagacatcaattttgcgtccctggctccgcggcacggcacgcggccgttcatgggcacctggaacgagatcggcaccagccagctgaacgggggcgccttcaattggagcagtgtctggagcgggcttaaaaatttcggctcgacgctccggacctatgggaacaaggcctggaatagtagcacggggcagttgttaagggaaaagctcaaagaccagaacttccagcagaaggtggtggacgggctggcctcgggcattaacggggtggtggacatcgcgaaccaggccgtgcagcgcgagataaacagccgcctggacccgcggccgcccacggtggtggagatggaagatgcaactcttccgccgcccaaaggcgagaagcggccgcggcccgacgcggaggagacgatcctgcaggtggacgagccgccctcgtacgaggaggccgtcaaggccggcatgcccaccacgcgcatcatcgcgccgctggccacgggtgtaatgaaacccgccacccttgacctgcctccaccacccacgcccgctccaccgaaggcagctccggttgtgcaggcccctccggtggcgaccgccgtgcgccgcgtccccgcccgccgccaggcccagaactggcagagcacgctgcacagtatcgtgggcctgggagtgaaaagtctgaagcgccgccgatgctattgagagaggaaagaggacactaaagggagagcttaacttgtatgtgccttaccgccagagaacgcgcgaagatggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccatgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagtttggcccccaagggcgcccccaactccagtcagtggactgacaaagaacggcaaaatggtggacaaccacccactacaaaagatgttacaaaaacattcggagtagcagccaggggagggcttcatattactgataaaggactacaaataggagaagatgaaaataacgaggatggtgaagaagagatatatgcagacaaaactttccagccagaacctcaagtaggagaggaaaactggcaagatactgatgttttctatggcggcagagcgcttaaaaaggaaaccaaaatgaaaccatgctatggctcttttgccagacctaccaatgaaaaaggaggtcaagctaaatttttaaatggcgaaaacggtcaaccttctaaagatcaagatattacattagctttctttgatcttaaacaaaatgacactggaactactcaaaaccagccagatgttgtcatgtacactgaaaatgtgtatctggaaaccccagacacccatgtggtgtacaaacctggcaaggaagatacaagctccgctgctaaccttacacaacagtccatgcccaacaggcccaactacattggtttcagggacaactttgtggggctcatgtattacaacagcactggcaacatgggtgtgctggctggtcaggcctctcagttgaatgctgtggttgacttgcaagacagaaacaccgagctgtcttatcagctcttgctagattctctgggtgacagaaccagatactttagcatgtggaattctgcggtggacagctatgatccagatgtcaggatcattgagaatcacggtgttgaagatgagcttccaaattattgcttcccactggatggatctggcagcaataccgcatatcaaggtgttaaatatgaaaacggagctggcaatggaagctggaaagtagatggcgaagttgcttctcagaatcagatcgccaagggtaatctgtatgccatggagataaaccttcaggccaacctgtggaagagttttctgtactcgaacgtggcgctgtatctaccagactcctacaagtacacgccggccaacatcacgctgcccaccaacaccaacacctacgagtacatgaacggccgcgtggtggcaccctcgctggtggatgcctatgtcaacatcggtgcccgctggtcgctggaccccatggacaacgtcaaccccttcaaccaccaccgcaacgcgggtctgcgctaccgctccatgcttctgggcaacggccgctacgtgcccttccacatccaagtgccccaaaagttctttgccatcaagaacctgctcctgcttcccggttcctacacctacgagtggaacttccgcaaggatgtcaacatgatcctgcaaagttccctcggcaacgacctgcgcgtcgacggcgcctccgtccgcttcgacagcgtcaacctctatgccaccttcttccccatggcgcacaacaccgcctccacccttgaagccatgctgcgcaacgacaccaacgaccagtccttcaacgactacctctcggccgccaacatgctctacccaatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccattccctacctcgacggaaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgactaaggactggttcctcgtccagatgctctctcattacaacattggctaccagggcttctacgtgcctgacggttacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgcaccagccgcaccgcggcgtcatcgaggccgtctacctgcgcacgcccttctccgccggaaacgccaccacataagcatgagcggctccagcgaacgagagctcgcggccatcgtgcgcgacctgggctgcgggccctactttttgggaacccacgacaagcgcttccctggcttcctcgccggcgacaagctggcctgcgccatcgtcaacacggccggccgcgagaccggaggcgtgcactggctcgccttcggctggaacccgcgctcgcgcacctgctacatgttcgacccctttgggttctcggaccgccggctcaagcagatttacagcttcgagtacgaggccatgctgcgccgcagcgccctggcctcctcgcccgaccgctgtctcagcctcgagcagtccacccagaccgtgcaggggcccgactccgccgcctgcggacttttctgttgcatgttcttgcatgccttcgtgcactggcccgaccgacccatggacgggaaccccaccatgaacttgctgacgggggtgcccaacggcatgctacaatcgccacaggtgctgcccaccctccggcgcaaccaggaggagctctaccgcttcctcgcgcgccactccccttactttcgatcccaccgcgccgccatcgaacacgccaccgcttttgacaaaatgaaacaactgcgtgtatctcaataaacagcacttttattttacatgcactggagtatatgcaagttatttaaaagtcgaaggggttctcgcgctcgtcgttgtgcgccgcgctggggagggccacgttgcggtactggtacttgggaagccacttgaactcggggatcaccagtttgggaaccggaatctcggggaaggtctcgctccacatgcgccggctcatctgcagggcgcccagcatgtccggggcggagatcttgaaatcgcagttgggaccggtgctctgcgcgcgcgagttgcggtacacggggttgcagcactggaacaccatcagactggggtacttcacactggccagcacgctcttgtcggtgatctgatccttgtcaaggtcctcggcgttgctcaggccaaacggggtcatcttgcacagctggcggcccaggaagggcacgctctgaggcttgtggttacactcgcagtgaatgggcattagcatcatccccgcgccgcgctgcatattcgggtagagggccttgacaaaggccgagatctgtttgaaagcttgctgggccttggctccctcgctgaaaaacagcccgcagctcttcccgctgaactggttattcccgcaaccggcatcctgcacgcagcagcgcgcgtcatggctggtcagttgcaccacgctccgtccccagcggttctgggtcaccttggccttgctgggttgctccttcagcgcgcgctgtccgttctcgctggtcacatccatctccaccacgtggtctttgtggatcatcaccgttccatgcagacacttgagctggccttccacctcggtgcagccgtgatcccacagggcgcatccggtgcactcccagttcttatgcgcgatcccgctgtggctgaagatgtaaccttgcaacaggcgacccatgacggtgctaaaggctttctgggtggtgaaggtcagttgcagaccgcgggcctcctcgttcatccaggtctggcacatcttctggaagatctcggtctgctctggcatgagcttgtaagcatcgcgcaggccgctgtcgacgcggtagcgttccatcagcacgttcatggtatccatgcccttctcccaggacgagaccagaggcagactcagggggttgcgcacgttcaggacaccgggggtcgcgggctcgacgatgcgttttccgtccttgccttccttcaacagaaccggaggctggctgaatcccactcccacgatcacggcgtcttcctggggcatctcttcgtctgggtctacctttgtcacatgcttggtctttctggcttgcttcttttttggagggctgtccacggggaccacgtcctcctccgaagacccggagcccacccgctgatactttcggcgcttggtgggcagaggaggtggcggcgaggggctcctctcctgctccggcggatagcgcgccgacccgtggccccggggcggartggcctctcgctccatgaaccggcgcacgtcctgactgccgccggccattgtttcctaggggaagatggaggagcagccgcgtaagcaggagcaggaggaggacttaaccacccacgagcaacccaaaatcgagcaggacctgggcttcgaagagccggctcgtctagaacccccacaggatgaacaggagcacgagcaagacgcaggccaggaggagaccgacgctgggctcgagcatggctacctgggaggagaggaggatgtgctgctgaaacacctgcagcgccagtccctcatcctccgggacgccctggccgaccggagcgaaacccccctcagcgtcgaggagctgtgtcgggcctacgagctcaacntcttctcgccgcgcgtgccccccaaacgccagcccaacggcacctgcgagcccaacccgcgtctcaacttctatcccgtctttgcggtccccgaggcccttgccacctatcacatctttttcaagaaccaaaagatccccgtctcctgccgcgccaaccgcacccgcgccgacgcgctcctcgctctggggcccggcgcgcgcatacctgatatcgcttccctggaagaggtgcccaagatcttcgaagggctcggtcgggacgagacgcgcgcggcgaacgctctgaaagaaacagcagaggaagagggtcacactagcgccctggtagagttggaaggcgacaacgccaggctggtcgtgctcaagcgcagcgtcgagctcacccacttcgcctaccccgccgttaacctcccgcccaaggtcatgcgtcgcatcatggatcagcttatcatgccccacatcgaggccatcgatgagacccaagagcagcgccccgaggacgcccggcccgtggtcagcgacgagatgctcgcgcgctggctcgggacccgcgacccccaggctttggaacagcggcgcaagctgatgctggccgtagtcctggtcaccctcgagctcgaatgcatgcgccgcttcttctgcgaccccgagaccctgcgcaaggtcgaggagaccctgcactacactttcagacacggtttcgtcaggcaagcctgcaagatctccaacgtggagctgaccaacctggtctcctgcctggggatcctgcatgagaaccgcctggggcagacagtgctccactctaccctgaagggcgaggcacggcgggactatgtccgcgactgcgtctttctctttctatgccacacatggcaagcagccatgggcgtgtggcagcagtgtctcgaggacgagaacctgaaggagctggacaagcttcttgctagaaaccttaaaaagttgtggacgggcttcgacgagcgcaccgtcgcctcggacctggccgagatcgttttccccgagcgcctgaggcatacgctgaaaggcgggctgcccgacttcatgagccagagcatgttgcaaaactaccgcactttcattctcgagcgctcgggtatcctgcccgccacctgcaacgccttcccctccgactttgtcccgctgagctaccgcgagtgtcccccgccgctgtggagccactgctacctcttgcagctggctaactacatctcctaccactcggacgtgatcgaggacgtgagcggcgaggggctgctcgagtgccactgccgctgcaacctgtgctccccgcaccgctccctggtctgcaacccccagctcctgagcgagacccaggtcatcggtaccttcgagctgcaaggtccggagaagtccaccgctccgctgaaactcacgccggggttgtggacttccgcgtacctgcgcaaatttgtacccgaagactaccacgcccatgagataaagttcttcgaggaccaatcgcgtccgcagcacgcggatctcacggcctgcgtcatcacccagggcgcgatcctcgcccaattgcatgccatccaaaaatcccgccaagagtttcttctgaaaaagggtagaggggtctacctggacccccagacgggcgaggtgctcaacccgggtctcccccagcatgccgaggaagaagcaggagccgctagtggaggagatggaagaagaatgggacagccaggcagaggaggacgaatgggaggaggagacagaggaggaagaattggaagaggtggaagaggagcaggcaacagagcagcccgtcgccgcaccatccgcgccggcagccccggcggtcacggatacaacctccgctccggtcaagcctcctcgtagatgggatcgagtgaagggtgacggtaagcacaagcggcagggctaccgatcatggagggcccacaaagccgcgatcatcgcctgcttgcaagactgcggggggaacatcgctttcgcccgccgctacctgctcttccaccgcggggtgaacatcccccgcaacgtgttgcattactaccgtcaccttcacagctaagaaaaagcaagtaagaggagtcgccggaggaggcctgaggatcgcggcgaacgagccctcgaccaccagggagctgaggaaccggatcttccccactctttatgccatttttcagcagagtcgaggtcagcagcaagagctcaaagtaaaaaatcggtctctgcgctcgctcacccgcagttgcttgtaccacaaaaacgaagatcagctgcagcgcactctcgaagacgccgaggctctgttccacaagtactgcgcgctcactcttaaagactaaggcgcgcccacccggaaaaaaggcgggaattacctcatcgccaccatgagcaaggagattcccaccccttacatgtggagctatcagccccagatgggcctggccgcgggcgcctcccaggactactccacccgcatgaactggctcagtgccggcccctcgatgatctcacgggtcaacggggtccgtaaccatcgaaaccagatattgttggagcaggcggcggtcacatccacgcccagggcaaagctcaacccgcgtaattggccctccaccctggtgtatcaggaaatccccgggccgactaccgtactacttccgcgtgacgcactggccgaagtccgcatgactaactcaggtgtccagctggccggcggcgcttcccggtgcccgctccgcccacaatcgggtataaaaaccctgatgatccgaggcagaggcacacagctcaacgacgagttggtgagctcttcgatcggtctgcgaccggacggagtgttccaactagccggagccgggagatcatccttcactcccaaccaggcctacctgaccttgcagagcagctcttcggagcctcgctccggaggcatcggaaccctccagttcgtggaggagtttgtgccctcggtctacttcaaccccttctcgggatcgccaggcctctacccggacgagttcataccgaacttcgacgcagtgagagaagcggtggacggctacgactgaatgtcccatggtgactcggctgagctcgctcggttgaggcatctggaccactgccgccgcctgcgctgcttcgcccgggagagctgcggactcatctactttgagctgcccgaggagcaccccaacggccctgcacacggagtacggatcaccgtagagggcaccgccgagtctcacctggtcaggttcttcacccagcaacccttcctggtcgagcgggaccggggcgccaccacctacaccgtctactgcatctgtcctaccccaaagttgcatgagaatttttgctgtactctttgtggtgagtttaataaaagctgaactaagaacctactttggaatcccttgtcgtcatcaaatccacaagaccatcaacttcacctttgaggaacaggtgaactttacctgcaagccacacaagaagtacgtcacctggttttaccagaacactactctagcagtagccaacacctgctcgaacgacggtgttcttcttccaaacaatctcaccagtggactaactttctcagtgaaaagggcaaagctaattcttcatcgccctattgtagaaggaacttaccagtgtcagagcggaccttgcttccacagtttcactttggtgaacgttaccggcagcagcacagtcgctccagaaactaaccttctttctgatactaacactcctaaaaccggaggtgagctctgggttccctctctgacagaggggggtagtcatattgaagcggtcgggtatttgattttaggggtggtcctgggtgggtgcatagcggtgctatattaccttccttgctgggtcgaaatcagggtatttatctgctgggtcagacattgtggggaggaaccatgaaggggctcttgctgattatcctttccctggttgggggtttactggcctgccacgaacagccacgatgtaacatcaccacaggcaatgagaggaacgactgctctgtagtgatcaaatgcgagcaccagtgtcctctcaacattacattcaagaataagaccatgggaaatgtatgggtgggattctggcaaccaggagatgagcagaactacacggtcactatccatggtagcgatggaaatcacactttcggtttcaaattcatttttgaagtcatgtgtgatatcacactgcatgtggctagacttcatggcttgtggccccctaccaaggagaacatggttgggttttctttggcttttgtgatcatggcctgtgcaatgtcaggtctgctggtaggggctctagtgtggttcctgaagcgcaagcccaggtacggaaatgaggagaaggaaaaattgctataaatctttttctcttcgcagaaccatgaatactttgaccagtgtcgtgctgctctctcttttagttattaatgtggaatgtgccgatcctattctagttagtgtagattggggaaaaaatcttacattagagggtcctaaagaaacaccagttgaatggtggggtggaagaaacatacaacaactgtgcatagggaatcaaaccaaacataaagagctaagtcacagatgtaatgtccagaacataactttactgtttgtaaatactagttttaatggagactactttgggtttaaaaatgataacagcggtatgaaacattataaagtcacagttataccccctaaaccctccactcggaaacctctttctcctccacactatgtaaacgcaactatggggcaaaacctaacattagtggggcctgcaaacattccagttacttggcttagtgaatatggcacgttgtgtgagggcaaaaaaattttgcacaaagnaattaaatcacacctgtaacgaacagaacctcacgttactgtttgttaatatgacacacaacggaccatattttggctttgacaaatacaacattgatagagagcagtatgaggtttctattattagtttgtttaaagttggcgctggacagaagaaaattgggaaaggacagaaaaaggaggaaaagacaaaaccaaactctagtgatttgggacaaagacaatccagaccaaagaaaaaagatattgttgaagaggtccaaatcaaaacaggagaaaatcgaacccttgttggtccacctggaaaagttgattggattaaactttccagtggaaacaataatgttcttaagttgtgtaatggcgacaagtatattaaacacacatgtgatggtcaaaatttaacattaattaatgtgactagaatttatgacggaacttattatggttctagcaatgatggctcaagtcattacaaagttaccatctatgaattacacaaagttaataaaactaaatctatgcttaagccatacactacaaaaagaactacagtgaatgcaacagatgacagtgctcacaaaattgctttgcagcaggaaaataatgggcaaacagaaaatgatcaagaatcaaaaattccatctgctactgtggcaatcgtggtgggagtgattgcgggcttcataactataatcattgtcattctgtgctacatctgctgccgcaagcgtcccagggcatacaataatatggtagacccactactcagcttctcttactgagactcagtcactttcatttcagaaccatgaaggctttcacagcttgcgttctgattagcataattacacttagtttagcagcacctaaaccagaagtatatacacaagttaatgtcactaggggtgggaatgctacactagatggaccatttaacaataacacatggacaagatatcatgatgatgggagaaaaaacggatggatgaatatttgtaaatggtcagacccatcatacacatgtcatagtaatggaagccttagtatttttgctttcaacattagttcaggtaaatataaagttcaaagttacactaacagttataatggattagatggttatgaaaaacttgaagttaaaatgtttaatctaacagtaattgagcctccaaccactagagcacccaccacagttaggacaactaaggaaacaacacagcctaccactgtacccactacacatccaaccaccacagtcagtacaactattgagaccactactcatactacacagctagacacaacagtgcagaatactactttactgattgaatttttactaagagggaatgaaagtactactgatcagacagaggctacctcaagtgccttcagcagtactgcaaatttaacttcgcttgcttggactaatgaaaccggagtatcattgatgcatggccagccttactcaggtttggatattcaaattacttttctggttgtctgtgggatctttattcttgtggttcttctgtactttgtctgctgcaaagccagagagaaatctagtaggcccatctacaggccagtaatcggggaacctcagcctctccaagtggaagggggtctaaggaatcttctcttctctttttcagtatggtgatcagccatgattcctaggttcttcctatttaacatcctcttctgtctcttcaacatctgcgctgccttcgcggccgtctcgcacgcctcgcccgactgtctcgggcccttccccacctacctcctctttgccctgctcacctgcacctgcgtctgcagcattgtctgcctggtcgtcaccttcctgcagctcatcgactggtgctgcgcgcgttacaattatctccaccacagtcccgaatacagggacaagaacgtagccagaatcttaaggctcatctgaccatgcagactctgctgatactgctatccctcctctcccctgcccttgctgactgtaaatttgcggacatatggaatttcttagactgttatcaagagaaaatggatatgccttcctattacttggtgattgtgggtgtagtcatggtctgctcctgcactttctttgctatcatgatctacccctgttttgatctcggctggaactctgttgaggcattcacatacacactagaaagcagttcactagcctccacgccgccacccacaccgcctccccgcagaaatcagttccccctgattcagtacttagaagagccccctccccggcccccttccactgttagctactttcacataaccggcggcgatgactgaccaccacctggacctcgagatggacggccaggcctccgagcagcgcatcctgcaactgcgcgtccgtcagcagcaggagcgggccgccaaggagctcctcgatgccatcaacatccaccagtgcaagaagggcatcttctgcctggtcaaacaggcaaagatcacctacgagctcgtgtccaacggcaaacagcatcgcctcacctatgagatgccccagcagaagcagaagttcacctgcatggtgggcgtcaaccccatagtcatcacccagcagtcgggcgagaccagcggctgcatccactgctcctgcgaaagccccgagtgcatctactccctcctcaagaccctttgcggacttcgcgacctcctccccatgaactgattgattaaagcccagaaaccaatcaaacccccttccccatcaccccaaataacaatcattggaaataatcattcaataaagatcacttacttgaaatctgaaagtatgtctctggtgtagttgttcagcagcacctcggtaccctcctcccagctctggtactccagtccccggcgggcggcgaacttcctccacaccttgaaagggatgtcaaattcctggtccacaattttcattgtcttccctctcagatgtcaaagaggctccgggtggaagatgacttcaaccccgtctacccctatggctacgcgcggaatcagaatatccccttcctcactcccccctttgtctcctccgatggattccaaaacttcccccctggggtcctgtcactcaaactggctgatccaatcgccatcgccaatgggaatgtctcactcaaggtgggagggggactcactgtagaacaacagtctggaaaactgagtgtggatactaaggcacccttgcaagttgcaaatgacaacaaattggagctatcttatgatgatccatttaaggtagagaataacaaacttggaattaaagctggccatggtttagcagttgtaactaaagaaaacacaagtcttcctagtctagttggaacacttgtagttttaactggaaaaggaataggtactggatcaagtgcacatggaggaactattgatgtaagacttggtgaaggaggtgggttatcatttgatgaaaaaggagacttagtagcttgggacaaaaaaaatgatacacgcaccctttggacaacacctgtccttctccaaattgcaaagttgaaacagcaagagactcaaagctaaccttagcacttacaaaatgtggtagtcaaattttggccactgtatctttacttgttgttacgggcaaatatgctattataagtgacacagtcaacccaaagcagttctctattaagttactgtttaatgacaagggtgttttgttaagkgactcaaatcttgatgggacatattggaactatagaagcaacaataacaacataggcactccttataaagaggctgttggttttatgccaagcacaacagcttatcctaagccaaccaacaacaccagcacagatccggataaaaaagtgagtcaaggtaaaaataaaattgtaagcaatatttatcttggaggagaggtatatcaaccaggatttattgttgttaaatttaatcaggaaactgatgccaattgtgcatactctattacatttgattttggatggggtaaggtgtataaggatcctataccatatgatacctcttcttttactttctcatatatcgctcaagaatgaaagaccaataaacgtgtttttcattgaaaattttcatgtatctttattgatttttacaccagcacgggtagtcagtctcccaccaccagcccatttcacagtgtaaacaattctctcagcacgggtggccttaaataggggaatgttctgattagtgcgggaactggatttagtgtctataatccacacagtttcctggcgagccaaacggggatcggtgattgagatgaagccgtcctctgaaaagtcttccaagcgggcctcacagtccaaggtcacagtctggtggaatgagaagaacgcacagattcatactcggaaaacaggatgggtctgtgcctctccatcagcgccctcaacagtctctgccgccggggctcggtgcggctgctgcagatgggatcgggatcgcaagtctctctgactatgatccccacagccttcagcatcagtctcctggtgcgtcgggcacagcacctcatcctgatctcgctcatgttctcacagtaagtgcagcacataatcaccatgttattcagcagcccataattcagggtgctccagccaaagctcatgttggggatgatggaacccacgtgaccatcataccaaatgcggcagtatatcaggtgcctgcccctcatgaacacactgcccatatacatgatctctttgggcatgtttctgttcacaatctggcggtaccaggggaagcgctggttgaacatgcacccgtaaatgactctcctgaaccacacggccagcarggtgcctcccgcccggcactgcagggagcccggggacgaacagtggcaatgcaggatccagcgntcgnacccgctcaccatctgagctctcaccaagtccagggtagcggggcacaggcacactgacatacatctttttaaaatttttatttcctctggggtcaggatcatatcccaggggactggaaactcttggagcagggtaaagccagcagcacatggtaatccacggacagaacttacattatgatattcagcatgatcacaatcgggcagcagggggtgttgttcagtcagtgaggccctggtctcctcctcagatcgtggtaaacgggccctgcggtatggatgatggcggagcgaggtcgattgttcctcggtgctcattgtagtgcaccctcttgcgtaccttgtcgtacttctgccagcagaaagtggcccgggaacagcagatacccctcctccgtccgtcctttcgctgctgccgctcagtcatccaactgaagtacatccattcccgaaggttctggagaagttcctctgcatctgatgaaacaaaaagcccgtccatgcgaattcccctcatcacatcagccaggactctgtaggccatccccatccagttaatgctgccttgtctatcattcagagggggcggtggcaggattggaagaaccattatttttttactccaaacggtcgcgcagcaatataaaattcaggtcacggaggtggcacctctctcctccactgttttggtggaaacagacagccaaatcaaaaattatgcgattctcaaggtgctctactgtggcttccagcagaggctccacacgtacatccagaaacaacagcacattaaaagcgggcccgccatcctgctcmtcaatcatcatattacagtcctgaaccatccccaggtaattttcgtttttccagccttgaattatcgstacaa

SEQ ID NO: 30 - DNA, 2775 bp, Adenovirus type 36
  Adenovirus type 36 (Ad-36) hexon protein
atggccaccccctcgatgatgccgcagtgggcgtacatgcacatcgccgggcaggacgcctcggagtacctgagcccgggtctggtgcagtttgcccgcgccaccgacacgtacttcagcctgggcaacaagtttaggaaccccacggtggccccgacccatgatgtgaccacggaccggtcccagcgtctgacgctgcgcttcgtgcccgtggatcgcgaggacaccacgtactcgtacaaggcgcgcttcactctggccgtgggcgacaaccgggtgctagacatggccagcacgtactttgacatccgcggcgtcctggaccgcggtcccagcttcaaaccctactcgggcacggcttacaacagtttggcccccaagggcgcccccaactccagtcagtggactgacaaagaacggcaaaatggtggacaaccacccactacaaaagatgttacaaaaacattcggagtagcagccaggggagggcttcatattactgataaaggactacaaataggagaagatgaaaataacgaggatggtgaagaagagatatatgcagacaaaactttccagccagaacctcaagtaggagaggaaaactggcaagatactgatgttttctatggcggcagagcgcttaaaaaggaaaccaaaatgaaaccatgctatggctcttttgccagacctaccaatgaaaaaggaggtcaagctaaatttttaaatggcgaaaacggtcaaccttctaaagatcaagatattacattagctttctttgatcttaaacaaaatgacactggaactactcaaaaccagccagatgttgtcatgtacactgaaaatgtgtatctggaaaccccagacacccatgtggtgtacaaacctggcaaggaagatacaagctccgctgctaaccttacacaacagtccatgcccaacaggcccaactacattggtttcagggacaactttgtggggctcatgtattacaacagcactggcaacatgggtgtgctggctggtcaggcctctcagttgaatgctgtggttgacttgcaagacagaaacaccgagctgtcatatcagctcttgctagattctctgggtgacagaaccagatactttagcatgtggaattctacggtggacagctatgatccagatgtcaggatcattgagaatcacggtgttgaagatgagcttccaaattattgcttcccactggatggatctggcagcaataccgcatatcaaggtgttaaatatgaaaacggagctggcaatggaagctggaaagtagatggcgaagttgcttctcagaatcagatcgccaagggtaatctgtatgccatggagataaaccttcaggccaacctgtggaagagttttctgtactcgaacgtggcgctgtatctaccagactcctacaagtacacgccggccaacatcacgctgcccaccaacaccaacacctacgagtacatgaacggccgcgtggtggcaccctcgctggtggatgcctatgtcaacatcggtgcccgctggtcgctggaccccatggacaacgtcaaccccttcaaccaccaccgcaacgcgggtctgcgctaccgctccatgcttctgggcaacggccgctacgtgcccttccacatccaagtgccccaaaagttctttgccatcaagaacctgctcctgcttcccggttcctacacctacgagtggaacttccgcaaggatgtcaacatgatcctgcaaagttccctcggcaacgacctgcgcgtcgacggcgcctccgtccgcttcgacagcgtcaacctctatgccaccttattccccatggcgcgcaacaccgcctccacccttgaagccatgctgcgcaacgacaccaacgaccagtccttcaacgactacctctcggccgccaacatgctctacccaatcccggccaaggccaccaacgtgcccatctccatcccctcgcgcaactgggccgccttccgcggctggagtttcacccggctcaagaccaaggaaactccctccctcggctcgggtttcgacccctactttgtctactcgggctccattccctacctcgacggaaccttctacctcaaccacaccttcaagaaggtctccatcatgttcgactcctcggtcagctggcccggcaacgaccggctgctcacgccgaacgagttcgagatcaagcgcagcgtcgacggggagggctacaacgtggcccaatgcaacatgactaaggactggttcctcgtccagatgctctctcattacaacattggctaccagggcttctacgtgcctgagggttacaaggaccgcatgtactccttcttccgcaacttccagcccatgagcaggcaggtggtcgatgagatcaactacaaggactacaaggccgtcaccctgcccttccagcacaacaactcgggcttcaccggctacctcgcacccaccatgcgtcaggggcagccataccccgccaacttcccctacccgctcatcggccagacagccgtgccctccgtcacccagaaaaagttcctctgcgacagggtcatgtggcgcatccccttctccagcaacttcatgtccatgggcgccctcaccgacctgggtcagaacatgctctacgccaactcggcccacgcgctcgacatgaccttcgaggtggaccccatggatgagcccaccctcctctatcttctcttcgaagttttcgacgtggtcagagtgaacgccaccacataa

SEQ ID NO: 31 - DNA, 1089 bp, Adenovirus type 36
  Adenovirus type 36 (Ad-36) fiber coat protein
atgtcaaagaggctccgggtggaagatgacttcaaccccgtctacccctatggctacgcgcggaatcagaatatccccttcctcactcccccctttgtctcctccgatggattccaaaacttcccccctggggtcctgtcactcaaactggctgatccatgtctcactcaaggtgggagggggactcactgtagaacaacagtctggaaaactgagtgtggatactaaggcacccttgcaagttgcaaatgacaacaaattggagctatcttatgatgatccatttaaggtagagaataacaaacttggaattaaagctggccatggtttagcagttgtaactaaagaaaacacaagtcttcctagtctagttggaacacttgtagttttaactggaaaaggaataggtactggatcaagtgcacatggaggaactattgatgtaagacttggtgaaggaggtgggtatcatttgatgaaaaaggagacttagtagcttgggacaaaaaaaatgatacacgcaccctttggacaacacctgatccttctccaaattgcaaagttgaaacagcaagagactcaaagctaaccttagcacttacaaaatgtggtagtcaaattttggccactgtatctttacttgttgttacgggcaaatatgctattataagtgacacagtcaacccaaagcagttctctattaagttactgtttaatgacaagggtgttttgttaagtgactcaaatcttgatgggacatattggaactatagaagcaacaataacaacataggcactccttataaagaggctgttggttttatgccaagcacaacagcttatcctaagccaaccaacaacaccagcacagatccggataaaaaagtgagtcaaggtaaaaataaaattgtaagcaatatatcttggaggagaggtatatcaaccaggatttattgttgttaaatttaatcaggaaactgatgccaattgtgcatactctattacatttgatttggatggggtaaggtgtataaggatcctataccatatgatacctcttctactttctcatatatcgctcaagaatga

SEQ ID NO: 32 - DNA, 20 bp, Artificial Sequence
  synthetic Ad-36 fiber protein forward primer for SYBRgreen method
taagccaaccaacaacacca

SEQ ID NO: 33 - DNA, 20 bp, Artificial Sequence
  synthetic Ad-36 fiber protein reverse primer for SYBRgreen method
tgcacaattggcatcagttt

SEQ ID NO: 34 - DNA, 20 bp, Artificial Sequence
  synthetic Ad-36 fiber protein forward primer for SYBRgreen method
ggccatggtttagcagttgt

SEQ ID NO: 35 - DNA, 20 bp, Artificial Sequence
  synthetic Ad-36 fiber protein reverse primer for SYBRgreen method
gtccaaagggtgcgtgtatc

SEQ ID NO: 36 - DNA, 22 bp, Artificial Sequence
  synthetic Ad-36 fiber protein forward primer for SYBRgreen method
ttaactggaaaaggaataggta

SEQ ID NO: 37 - DNA, 24 bp, Artificial Sequence
  synthetic Ad-36 fiber protein reverse primer for SYBRgreen method
ggtgttgttggttggcttaggata

SEQ ID NO: 38 - DNA, 24 bp, Artificial Sequence
  synthetic Ad-36 fiber protein forward primer for Taqman method
caatgggaatgtctcactcaaggt

SEQ ID NO: 39 - DNA, 22 bp, Artificial Sequence
  synthetic Ad-36 fiber protein reverse primer for Taqman method
agggtgccttagtatccacact

SEQ ID NO: 40 - DNA, 22 bp, Artificial Sequence
  synthetic Ad-36 fiber protein primer for Taqman method
cagttttccagactgttgttct

(19) United States
(12) Patent Application Publication
(10) Pub. No.: US 2012/0244187 A1
(43) Pub. Date: Sep. 27, 2012

(54) Adenovirus Infection In Animals

(76) Inventor: Richard L. Atkinson, Mechanicsville, VA (US)

(21) Appl. No.: 13/501,763

(22) PCT Filed: Oct. 22, 2010
(86) PCT No.: PCT/US2010/053817

§ 371 Date: Jun. 12, 2012

Related U.S. Application Data
(60) Provisional application No. 61/254,068, filed on Oct. 22, 2009.
(60) Provisional application No. 61/254,083, filed on Oct. 22, 2009.

(51) Int. Cl. C12Q 1/70; A61P 31/20; A61K 39/235

(52) U.S. Cl. 424/233.1; 435/5

(57) ABSTRACT

Animals have tested positive for unsuspected natural infection with lipogenic adenoviruses. Methods for testing animals, including food stuffs and experimental animals, for lipogenic adenovirus infection are disclosed. Exposure to infected meat and animal co-products may cause health and safety issues. As a result of lipogenic adenovirus infection in experimental animal species, research related to fat or glucose metabolism, energy metabolism, cancer biology, and obesity research may have been or may be negatively affected or compromised.

### CROSS-REFERENCES TO RELATED APPLICATIONS

This application claims priority to U.S. Provisional Application Ser. No. 61/254,083, filed on Oct. 22, 2009, and U.S. Provisional Application Ser. No. 61/254,068, filed on Oct. 22, 2009, the disclosures of which are herein expressly incorporated by reference in their entirety.

### BACKGROUND OF THE INVENTION

1. Field of the Invention

The invention relates generally to testing for unsuspected (natural) lipogenic adenovirus infection in animals, including food stuffs such as meat and animal edible co-products prior to consumption by humans and non-human animals, experimental animals prior to experimental use, and domestic animals.

2. Related Art

Recent studies have revealed that pathogenic hazards (i.e., salmonella, campylobacter, E. Coli, and the like) carried primarily by healthy animals as causing the majority of meat-borne risks to consumers. This has obvious implications for the implementation of food safety measures by the industry. In many situations, prevention and control of pathogenic hazards of public health importance are achieved in parallel of prevention and control of diseases and conditions of animal health importance. This duality of functions becomes especially important in a “production to consumption” approach to food control.

The Applicant has determined the prevalence of unsuspected (natural) infection with human adenovirus-36 (Ad-36) in domestic animals, such as poultry. This raises a number of concerns for meat packers, processors, and purchasers, as exposure to infected meat and animal edible co-products, such as milk, may lead to infection in the consumer, and gives rise to a number of health concerns.

For example, human adenovirus-36 (Ad-36) causes obesity in humans and non-human animals, and also causes or contributes to a number of diseases due to the complications of obesity and the outcome of regimens that affect body weight as Applicant has demonstrated in U.S. Pat. Nos. 7,442,511 and 7,507,418, the disclosures of which are incorporated by reference in their entirety. For example, complications of obesity may include, inter alia, diabetes mellitus, hypertension, hyperlipoproteinemia, cardiac disease such as atherosclerotic disease and congestive heart failure, pulmonary diseases such as sleep apnea and asthma, cerebrovascular accidents, cancers such as breast, uterus colon and prostate cancer, gall bladder disease such as stones and infection, toxemia during pregnancy, risks during surgery, gout, decreased fertility, degenerative arthritis, and early mortality.

In addition, the Applicant has discovered that unsuspected natural infection of lipogenic adenovirus, such as adenovirus type 36 (Ad-36), occurs in multiple experimental animal species. The consequences of these findings may be that a great deal of the research that has been done over the last 20-30 years related to obesity and/or metabolic function may be compromised by the presence of unsuspected Ad-36 infection.

Previous studies of animals deliberately infected with the lipogenic adenovirus showed that they gained significantly more body fat and body weight and had changes in metabolic functions compared to uninfected control animals. The effects of the lipogenic adenovirus are on metabolic rate because food intake did not change, but the animals gained fat and body weight. In all species of experimental animals the blood cholesterol and triglycerides were paradoxically lower in infected animals. Blood sugar and/or sensitivity to insulin were altered by the virus in animals and in tissue culture studies. Studies in tissue culture reveal that Ad-36 promotes malignant changes in cells, including breast epithelial cells, and alters a series of enzymes that are associated with cancer.

Therefore, in particular, research related to fat or glucose metabolism, energy metabolism, cancer biology, and obesity research may have been or may be negatively affected or compromised by using experimental animals positive for lipogenic adenovirus infection. This could represent millions of dollars of research that may need to be revisited or completely redone.

### SUMMARY OF THE INVENTION

The invention provides methods for evaluating the safety and/or suitability of animals for consumption or experimental use. These methods provide ways to limit consumer exposure to lipogenic adenoviruses from food stuffs and reduce/prevent infection to consumers. These methods also provide ways for investigators, who use experimental animals, to test for the presence of lipogenic adenovirus infection prior to using laboratory animals for experimental use. Embodiments of the invention may be implemented in a number of ways.

According to one aspect of the invention, a method for evaluating the safety and suitability of animals or meat for consumption by a consumer may include screening a sample from an animal designated for consumption to determine whether the animal is infected with a lipogenic adenovirus, and taking corrective action if the animals is positive for lipogenic adenovirus infection. The corrective action may include discarding infected meat, administering an antiviral agent (e.g., in a sufficient amount to significantly reduce or eliminate the adenovirus infection), disinfecting equipment, isolation of infected animals, treating infected animals, or emergency slaughter of infected animals. The method may also include taking corrective action if the animal is not infected with lipogenic adenovirus, which may include administering a lipogenic adenovirus vaccine to the animal prior to processing its meat for consumption.

The sample may be obtained and screened from the animal ante-mortem and/or post-mortem. The animal may include without limitation ungulates, solipeds, birds, lagomorphs, farmed game, farmed game birds, and wild game. The consumer may be a human or non-human animal. The lipogenic adenovirus may be adenovirus type 5, adenovirus type 36, or adenovirus type 37. The sample may be a biological sample, body fluid, a tissue sample, an organ sample, feces, blood, saliva, and any combination thereof.

The sample may be screened by screening for antibodies reactive to the lipogenic adenovirus in the sample. The antibodies may be reactive to a lipogenic adenovirus protein such as Ad-36 hexon protein and/or Ad-36 fiber coat protein. The antibodies may be reactive to one or more peptides encodes by the peptide sequences of SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, SEQ ID NO.: 15, SEQ ID NO.: 16, SEQ ID NO.: 17, and SEQ ID NO.: 18. The screening may be performed by using serum neutralization assay or ELISA.

Alternatively, the sample may be screened for the presence of lipogenic adenovirus nucleic acids. The nucleic acids may be nucleic acids encoding a hexon protein encoded by the nucleic acid sequence of SEQ ID No. 29. The nucleic acids may be nucleic acids encoding the fiber coat protein encoded by the nucleic acid sequence of SEQ ID No. 30. The nucleic acids may be detected using one or more nucleic acids including SEQ ID NO.: 19, SEQ ID NO.: 20, SEQ ID NO.:21, SEQ ID NO.:22, SEQ ID NO.:23, SEQ ID NO.:24, and SEQ ID NO.:25.

The screening step may also include screening for the presence of a biomarker associated with a lipogenic adenovirus infection in the sample. The biomarker may include fatty acid synthetase (FAS), peroxisome proliferator-activated receptor (PPAR) family proteins, CCAAT/enhancer-binding proteins (C/EBP), adipose tissue differentiation determination-dependent factor 1 (ADD-1)/sterol response element-binding protein (SREBP-1), glycerol-3-phosphdehydrogenase, and/or lipoprotein lipase.

According to another aspect of the invention, a method for evaluating the suitability of experimental animals or animal co-products for experimental use may include screening a sample from an animal designated for experimentation to determine whether the animal is infected with a lipogenic adenovirus, taking corrective action if the animals is positive for lipogenic adenovirus infection. The method may further comprise conducting research on animals that are not infected and/or segregating data from experiments based on the presence or absence of the adenovirus. If the animals is positive for lipogenic adenovirus infection, the animal may not be suitable for experiments directed to fat metabolism, glucose metabolism, energy metabolism, cancer biology, or obesity.

The experimental animals may include without limitation rats, guinea pigs, rabbits, non-human primates, humans, birds, cows, sheep, goats, and chickens. The animal co-products may include without limitation biological sample, blood, semen, saliva, serum, cerebral fluid, urine, and plasma.

Screening the sample may comprise screening for antibodies reactive to the lipogenic adenovirus in the sample. The antibodies may be reactive to a viral protein selected from the group consisting of Ad-36 fiber coat protein and Ad-36 hexon protein. Specifically, the antibodies may be reactive to one or more peptides encoded by the peptide sequences including SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, SEQ ID NO.: 15, SEQ ID NO.: 16, SEQ ID NO.: 17, and SEQ ID NO.: 18. The screening step may be performed by using at least one of serum neutralization assay and ELISA.

Alternatively, the screening step may comprise screening for lipogenic adenovirus nucleic acids. The nucleic acids may be nucleic acids encoding a hexon protein. In particular, the hexon protein may be encoded by the nucleic acid of SEQ ID No. 29. The nucleic acids may encode the fiber coat protein and may be encoded by the nucleic acid sequence comprising SEQ ID No.: 30. The nucleic acids may be detected using one or more nucleic acids including SEQ ID NO.: 19, SEQ ID NO.: 20, SEQ ID NO.:21, SEQ ID NO.:22, SEQ ID NO.:23, SEQ ID NO.:24, and SEQ ID NO.:25.

The lipogenic adenovirus may be adenovirus type 5, adenovirus type 36, and adenovirus type 37. The sample may be a biological sample, body fluid, a tissue sample, an organ sample, feces, blood, saliva, and any combination thereof.

The screening step may comprise screening for the presence of a biomarker associated with a lipogenic adenovirus infection in the sample. The biomarkers may include fatty acid synthetase (FAS), peroxisome proliferator-activated receptor (PPAR) family proteins, CCAAT/enhancer-binding proteins (C/EBP), adipose tissue differentiation determination-dependent factor 1 (ADD-1)/sterol response element-binding protein (SREBP-1), glycerol-3-phosphdehydrogenase, and lipoprotein lipase.

Additional features, advantages, and embodiments of the invention may be set forth or apparent from consideration of the following detailed description, and claims. Moreover, it is to be understood that both the foregoing summary of the invention and the following detailed description are exemplary and intended to provide further explanation without limiting the scope of the invention as claimed.

### DETAILED DESCRIPTION OF THE INVENTION

It is understood that the invention is not limited to the particular methodology, protocols, and reagents, etc., described herein, as these may vary as the skilled artisan will recognize. It is also to be understood that the terminology used herein is used for the purpose of describing particular embodiments only, and is not intended to limit the scope of the invention. It also is be noted that as used herein and in the appended claims, the singular forms “a,” “an,” and “the” include the plural reference unless the context clearly dictates otherwise. Thus, for example, a reference to “a cell” is a reference to one or more cells and equivalents thereof known to those skilled in the art.

Unless defined otherwise, all technical and scientific terms used herein have the same meanings as commonly understood by one of ordinary skill in the art to which the invention pertains. The embodiments of the invention and the various features and advantageous details thereof are explained more fully with reference to the non-limiting embodiments and examples that are described and/or illustrated in the accompanying drawings and detailed in the following description. It should be noted that the features illustrated in the drawings are not necessarily drawn to scale, and features of one embodiment may be employed with other embodiments as the skilled artisan would recognize, even if not explicitly stated herein. Descriptions of well-known components and processing techniques may be omitted so as to not unnecessarily obscure the embodiments of the invention. The examples used herein are intended merely to facilitate an understanding of ways in which the invention may be practiced and to further enable those of skill in the art to practice the embodiments of the invention. Accordingly, the examples and embodiments herein should not be construed as limiting the scope of the invention, which is defined solely by the appended claims and applicable law.

Accordingly, provided immediately below is a “Definition” section, where certain teems related to the invention are defined specifically for clarity, but all of the definitions are consistent with how a skilled artisan would understand these terms. Particular methods, devices, and materials are described, although any methods and materials similar or equivalent to those described herein can be used in the practice or testing of the invention. All references referred to herein are incorporated by reference herein in their entirety.

“Ad-2” is Adenovirus type 2.

“Ad-5” is Adenovirus type 5.

“Ad-31” is Adenovirus type 31.

“Ad-36” is Adenovirus type 36.

“Ad-37” is Adenovirus type 37.

PPAR is peroxisome proliferator activated receptors.

CEBP is CCAAT-enhancer binding protein.

FAS is fatty acid synthase.

PI3K is phosphatidylinositol 3-kinase.

“Lipogenic adenovirus,” as used herein, generally refers to adenoviruses that are capable of stimulating increase lipid production in cells, tissues, and/or organs by facilitating expression of lipogenic enzymes, which in turn produce excess fatty acids and promote fat storage. Moreover, lipogenic adenoviruses may cause or exacerbate malignant changes in cells. The lipogenic adenoviruses of the invention may include Ad-5, Ad-36, and Ad-37.

A “biological sample,” as used herein, generally refers to a sample of tissue or fluid from a human or animal including, but not limited to plasma, serum, spinal fluid, lymph fluid, the external sections of the skin, respiratory, intestinal and genitourinary tracts, tears, saliva, blood cells, tumors, organs, tissue and sample of in vitro cell culture constituents.

“Organ,” as used herein, generally refers to a tissue that performs a specific function or group of functions within an organism. An exemplary list of organs includes lungs, heart, blood vessels, blood, salivary glands, esophagus, stomach, liver, gallbladder, pancreas, intestines, rectum, anus, endocrine glands such as hypothalamus, pituitary or pituitary gland, pineal body or pineal gland, thyroid, parathyroids, adrenals, skin, hair, nails, lymph, lymph nodes, tonsils, adenoids, thymus, spleen, muscles, brain, spinal cord, peripheral nerves, nerves, sex organs such as ovaries, fallopian tubes, uterus, vagina, mammary glands, testes, vas deferens, seminal vesicles, prostate, and penis, pharynx, larynx, trachea, bronchi, diaphragm, bones, cartilage, ligaments, tendons, kidneys, ureters, bladder, and urethra.

“Organ system,” as used herein, generally refers to a group of related organs. Organ systems include, without limitation, circulatory system, digestive system, endocrine system, integumentary system, lymphatic system, muscular system, nervous system, reproductive system, respiratory system, skeletal system, and urinary system.

The term “antiviral agent” refers to a chemical material or compound which, when administered to an organism (human or animal) kills viruses, blocks virus effects on the host cells, prevents infection with a virus, and/or suppresses viral replication and, hence, inhibits the capability of the virus to infect, multiply, reproduce, or alter host physiology and biochemistry. Included are derivatives and analogs of those compounds or classes of compounds specifically mentioned that also induce the desired pharmacologic effect. In particular, the antiviral agent may encompass a single biological or abiological chemical compound, or a combination of biological and abiological compounds that may be required to cause a desirable therapeutic effect.

Antiviral agents are a class of compounds used specifically to treat viral infections. An antiviral agent kills viruses and/or suppresses their replication, thereby inhibiting their ability to multiply and reproduce. Antiviral agents may be useful in the early stages of some viral infections, or to prevent reoccurrences or reactivation in chronic infections. Most antiviral agents exert their effects only during a certain stage of viral replication. Antivirals are designed to target and disable viral proteins, or parts of proteins, that are specific to the virus, and ideally these viral targets are as distinct as possible from any proteins, or parts of proteins, in humans in order to reduce unacceptable side effects.

Antiviral agents have been developed to disable viral replication at different steps of the viral life cycle including, but not limited to, viral entry into the host cell, replication of the viral genome, activation of viral protein, and release of new virus particles. For example, antiviral agents may inhibit the ability of a virus to enter a host cell by preventing the virus from binding to receptors on the host cell that are required for entry, or blocking the virus uncoating process inside the host cell such that the virus cannot release its contents. Additionally, antiviral agents may target the processes that synthesize viral components after a virus invades a host cell (e.g., adenovirus DNA ploymerase). There are several classes of antiviral agents that block viral synthesis including ribonucleotide reductase inhibitors, nucleotide analogs, nucleoside analogs, antisense drugs, ribozymes, and protease inhibitors. Finally, antiviral agents, such as interferon drugs, may function to prevent the release of newly packaged virus particles from the infected host cell thereby blocking the final stage of the viral life cycle. Antiviral agents that block adenovirus replication at any of the stages in the adenovirus life cycle may be effective treatments for preventing lipogenic adenovirus related cancer or other lipogenic adenovirus related diseases.

Antiviral agents may act in several ways to inhibit proliferation of lipogenic adenoviruses in the treatment of lipogenic adenovirus related cancers and/or diseases. One possible mechanism is to prevent the viral infection altogether by preventing lipogenic adenoviral attachment to the host cell. Unless the lipogenic adenovirus actually infects an individual, it may not cause the lipogenic adenovirus related cancer and/or diseases in the individual. Another possible mechanism is that an antiviral agent may prevent the virus from being active within the host's cells. For lipogenic adenoviruses, such antiviral agents may act by several mechanisms and/or at one of several steps: (i) an antiviral agent may prevent the viral DNA from entering into the nucleus of the host cell thereby preventing the lipogenic adenovirus from causing the cancers and/or diseases associated with infection; (ii) an antiviral agent may block the viral DNA from activating the host's DNA to increase production of FAS; (iii) antiviral agents may block the transcription of DNA to RNA or translation of RNA to protein; (iv) antiviral agents may block the effects of the viral proteins that are made by the early genes of the virus, such as E4orfl or E1A; and/or (v) antiviral agents may prevent the viral-mediated inhibition of host cell and body defense mechanisms, thereby allowing the lipogenic adenovirus infected host cell to die or be destroyed.

Antiviral agents are well known in the art. A number of prescription agents have been shown to have antiviral effects, which may be used in combination with the antiviral agents of the invention. These antiviral agents include, without limitation: Abacavir, Acyclovir, Amantadine, Amprenavir, Cidofovir, Didanosine, Darunavir, Delavirdine, Didox, Efavirenz, Emtricitabine, Enfuvirtide, Entecavir, Famciclovir, Foscarnet, Gancyclovir, Gardasil, Indinavir, Lamivudine, Nevirapine, Nelfinavir, Oseltamivir, Palivizumab, Pleconaril, Ribavirin, Rimantadine, Ritonavir, Saquinavir, Stavudine, Tridox, Valacyclovir, Vidarabine, Zalcitabine, Zanamivir, and Zidovudine.

An “isolated” or “substantially pure,” nucleic acid (e.g., DNA, RNA, or a mixed polymer) for example, is one which is substantially separated from other cellular components which naturally accompany a native human or animal sequence or protein, e.g., ribosomes, polymerases, many other human or animal genome sequences and proteins. The term embraces a nucleic acid sequence or protein which has been removed from its naturally occurring environment, and includes recombinant or cloned DNA isolates and chemically synthesized analogs or analogs biologically synthesized.

The term “antibody,” as used herein generally refers to antibodies, digestion fragments, specified portions and variants thereof, including antibody mimetics or comprising portions of antibodies that mimic the structure and/or function of an antibody or specified fragment or portion thereof, including single chain antibodies and fragments thereof. The invention encompasses antibodies and antibody fragments capable of binding to a biological molecule (such as an antigen or receptor), such as the fiber coat protein of lipogenic adenoviruses, and specifically, Ad-36, or portions thereof.

The term “nucleic acid sequence,” as used herein generally includes an oligonucleotide, nucleotide, or polynucleotide, and fragments thereof. The term is not limited by length and is generic to linear polymers of polydeoxyribonucleotides (containing 2-deoxy-D-ribose), polyribonucleotides (containing D-ribose), and any other N-glycoside of a purine or pyrimidine base, or modified purine or pyrimidine bases. These terms include double- and single-stranded DNA, as well as double- and single-stranded RNA.

A nucleic acid, polynucleotide or oligonucleotide can comprise, for example, phosphodiester linkages or modified linkages including, but not limited to phosphotriester, phosphoramidate, siloxane, carbonate, carboxymethylester, acetamidate, carbamate, thioether, bridged phosphoramidate, bridged methylene phosphonate, phosphorothioate, methylphosphonate, phosphorodithioate, bridged phosphorothioate or sulfone linkages, and combinations of such linkages.

A nucleic acid, polynucleotide or oligonucleotide can comprise the five biologically occurring bases (adenine, guanine, thymine, cytosine and uracil) and/or bases other than the five biologically occurring bases. For example, a polynucleotide of the invention can contain one or more modified, non-standard, or derivatized base moieties, including, but not limited to, N⁶-methyl-adenine, N⁶-tert-butyl-benzyl-adenine, imidazole, substituted imidazoles, 5-fluorouracil, 5-bromouracil, 5-chlorouracil, 5-iodouracil, hypoxanthine, xanthine, 4-acetylcytosine, 5-(carboxyhydroxymethyl)uracil, 5-carboxymethylaminomethyl-2-thiouridine, 5-carboxymethylaminomethyluracil, dihydrouracil, beta-D-galactosylqueosine, inosine, N⁶-isopentenyladenine, 1-methylguanine, 1-methylinosine, 2,2-dimethylguanine, 2-methyladenine, 2-methylguanine, 3-methylcytosine, 5-methylcytosine, N⁶-methyladenine, 7-methylguanine, 5-methylaminomethyluracil, 5-methoxyaminomethyl-2-thiouracil, beta-D mannosylqueosine, 5′-methoxycarboxymethyluracil, 5-methoxyuracil, 2-methylthio-N-6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, uracil-5-oxyacetic acid methylester, 3-(3-amino-3-N²-carboxypropyl) uracil, (acp3)w, 2,6-diaminopurine, and 5-propynyl pyrimidine. Other examples of modified, non-standard, or derivatized base moieties may be found in U.S. Pat. Nos. 6,001,611; 5,955,589; 5,844,106; 5,789,562; 5,750,343; 5,728,525; and 5,679,785.

Furthermore, a nucleic acid, polynucleotide or oligonucleotide can comprise one or more modified sugar moieties including, but not limited to, arabinose, 2-fluoroarabinose, xylulose, and a hexose.

The term “fragment,” as used herein generally includes any portion of a heterologous peptide or nucleic acid sequence. Heterologous peptide fragments retain at least one structural or functional characteristic of the subject heterologous polypeptides. Nucleic acid sequence fragments are greater than about 60 nucleotides in length, and most preferably includes fragments that are at least about 100 nucleotides, at least about 1000 nucleotides, and at least about 10,000 nucleotides in length.

The term “PCR,” as used herein, generally refers to a method for amplifying, detecting, or quantifying a specific region of an analyte. One skilled in the art appreciates that there are several variations on the basic PCR technique such as allele-specific PCR, assembly PCR or polymerase cycling assembly (PCA), colony PCR, helicase-dependent amplification, hot start PCR, intersequence-specific (ISSR)PCR, inverse PCR, ligation-mediated PCR, methylation-specific PCR, multiplex ligation dependent probe amplification, multiplex PCR, nested PCR, overlap-extension PCR, quantitative PCR, quantitative real-time PCR, RT-PCR, thermal asymmetric interlaces (TAIL) PCR, touchdown PCR, and PAN-AC. Additionally, one skilled in the art would understand how to practice these variations on the basic PCR technique.

“Biomarker,” as used herein, generally refers to an organic biomolecule that is differentially present in a sample taken from a subject of one phenotypic status (e.g., adipose tissue hypertrophy) as compared with another phenotypic status (e.g., no adipose tissue hypertrophy). A biomarker is differentially present between different phenotypic statuses if the mean or median expression level of the biomarker in the different groups is calculated to be statistically significant. Common tests for statistical significance include, among others, t-test, ANOVA, Kruskal-Wallis, Wilcoxon, Mann-Whitney and odds ratio. Biomarkers, alone or in combination, provide measures of relative risk that a subject belongs to one phenotypic status or another. As such, they are useful as markers for disease (diagnostics), therapeutic effectiveness of a drug (theranostics), and for drug toxicity. The differential presence of a biomarker may include, for example, an expression level of at least 10%, 20%, 50%, 100%, 200%, 500% or more in the lipogenic adenovirus infected cell compared with the expression level of the biomarker endogenously expressed in the normal cell.

“Subject,” or “consumer,” as used herein, includes human and non-human animals.

“Contaminant,” as used herein, includes any biological or chemical agent, foreign matter or other substance not intentionally added to meat that may compromise meat safety or suitability. In certain embodiments of the invention, the contaminant may be a lipogenic adenovirus, such as Ad-36.

“Contamination,” as used herein, refers to the introduction or occurrence of a contaminant in meat of the meat environment.

“Corrective action,” as used herein generally refers to procedures followed as a result of a test or assay. The actions may include, disinfection, administration of an antiviral agent (e.g., in a sufficient amount to significantly reduce or eliminate the adenovirus infection), emergency slaughter, preventive or remedial measures, and/or notifying a competent authority. If the animal meat and/or edible co-product is found to be infected with lipogenic adenovirus, the meat and/or edible co-product may be irradiated, cooked, removed from meat processing, discarded, and/or the processing equipment disinfected.

“Disinfection,” as used herein generally refers to the reduction or elimination, by means of chemical agents and/or physical methods, of the number of pathogens (e.g., lipogenic adenoviruses) in the environment, to a level that does not compromise meat safety or suitability.

“Emergency slaughter,” as used herein, generally refers to the immediate slaughter of an animal for reasons of meat hygiene or animal welfare, or to prevent the spread of disease, such as lipogenic adenovirus infection.

“Meat hygiene,” as used herein, generally refers to all conditions and measures to ensure the safety and suitability of meat at all stages of the food chain.

“Meat safety,” as used herein generally refers to the assurance that meat will not cause harm to the consumer when it is prepared or eaten according to its intended use.

“Meat suitability,” as used herein, generally refers to the assurance that meat is acceptable for consumption according to its intended use.

“Preventive measures,” as used herein generally refers to the physical or chemical or other means that can be used to control an identified meat safety hazard, such as lipogenic adenovirus infection.

“Notifiable disease,” as used herein generally refers to a disease, such as lipogenic adenovirus infection, that must be reported to the competent authority when its existence is known or suspected.

“Animal edible co-product,” generally refers to a product produced from an animal suitable for consumption such as eggs or milk.

“Preventive measures” as used herein generally refers to the physical or chemical or other means that can be used to control an identified hazard or pathogen, such as lipogenic adenovirus infection.

The invention generally relates to methods for evaluating the safety and/or suitability of animals for consumption and animals for experimental use.

In one aspect, methods for testing meat and animal edible co-products prior to consumption by consumers for unsuspected lipogenic adenoviruses, and in particular, Ad-36 infection are provided. Lipogenic adenovirus are naturally communicable and infection may be transmitted by direct and indirect contact with infected animals and their secretions, including saliva, blood, urine, feces, milk and semen, aerosol droplet dispersion, infected animal by-products, swill containing scraps of meat or other animal tissue and fomites.

Exposure to lipogenic infected meat and animal edible co-products by the consumer may result in consumer lipogenic adenovirus infection. Reducing and/or preventing exposure to lipogenic adenoviruses in meat and edible co-products is essential because as the Applicant has demonstrated, lipogenic adenovirus infection causes or contributes to obesity, diseases associated with obesity, and the outcome of regimens that affect body weight, as disclosed in U.S. Pat. Nos. RE39,544, RE39,914, 7,442,511 and 7,507,418, the disclosures of which are incorporated by reference in their entirety. Complications of obesity or diseases associated with obesity may include diabetes mellitus, hypertension, hyperlipoproteinemia, cardiac disease such as atherosclerotic disease and congestive heart failure, pulmonary diseases such as sleep apnea and asthma, cerebrovascular accidents, cancers such as breast, uterus colon and prostate cancer, gall bladder disease such as stones and infection, toxemia during pregnancy, risks during surgery, gout, decreased fertility, degenerative arthritis, and early mortality.

According to principles of the invention, meat and animal edible co-products from domestic and wild game animals should be tested for lipogenic adenovirus prior to consumption to avoid infection. “Meat,” as used herein, generally refers to skeletal muscle and associated fat, organs, liver, skin, brains, bone marrow, kidneys, and lungs, suitable for consumer consumption. “Meat,” may include any of the “white” or “red” meats. The animals to be tested for lipogenic adenovirus include domestic and wild animals such as bovine (beef), bovine (veal), porcine (pork), ovine (sheep), llama/alpaca, chicken, turkey duck, antelope, alligator, buffalo, black bear, crocodile, elk, frog, iguana, kangaroo, ostrich, lamb, rabbit, rattlesnake, turtle, wild boar, turtle, yak, venison, goose, duck, guinea, pheasant, quail, squab, wild and domestic turkey, wild and domestic ungulates, wild and domestic solipeds, wild and domestic birds, wild and domestic lagomorphs, farmed game, farmed game birds, wild game, and fish. Additionally, animal edible co-products should also be tested for lipogenic adenovirus infection such as milk, eggs, and any products produced from these co-products (e.g., cheese).

The meat may be evaluated at various stages from the production to consumption food pathway to eliminate the risk of consumer exposure to lipogenic adenovirus infection. The exposure assessment may be carried out at various stages, such as production (animal farm), transport and processing (e.g., ante-mortem screening and/or post-mortem screening), retail and storage, and preparation. If lipogenic adenovirus infection is determined at any of stage of the production to consumption food pathway, corrective action suitable for that stage may be taken. Corrective action, as described above, may include preventive measures, discarding infected meat, administering an antiviral agent (e.g., in a sufficient amount to significantly reduce or eliminate the adenovirus infection), disinfecting or sterilization of equipment, barn, isolation pens, loading dock, or abattoir, isolation of infected animals, treatment of infected animals, and/or emergency slaughter of infected animals.

In another aspect, the invention relates generally to testing experimental animal species for lipogenic adenovirus infection, and more specifically, for adenovirus type 36 infection. The experimental animal species may include, without limitation, rats, guinea pigs, rabbits, non-human primates, birds, farm animals (e.g., horses, cows, chickens, goats, sheep), mice, and other mammals. Human Ad-36 affects biochemical functions and induces obesity in chickens, mice, rats, monkeys, and humans as disclosed in Applicant's U.S. Pat. Nos. RE39,544, RE39,914, 7,442,511 and 7,507,418, the disclosures of which are incorporated by reference in their entirety.

Experimental infection with Ad-36 has extensive effects on multiple biochemical and physiological variables in animals. Nasal or intraperitoneal inoculation into chickens, mice, rats, and monkeys induces increases in adipose tissue of 50% to 150% as compared to uninfected control animals. A high percentage (60%-100%) of infected animals become obese as defined as a visceral or total body fat greater than the 85^{th} percentile of the value in control animals. Both fat cell size and fat cell number are significantly increased in experimentally infected animals. Serum lipids, including triglycerides and total and LDL cholesterol, paradoxically decrease by about 30%; e.g.—reductions in serum cholesterol of 34 mg/dl in experimentally infected monkeys compared to uninfected controls. Glucose transport is enhanced in adipocytes and muscle cells in vitro and in vivo leading to a reduction in serum insulin levels in infected rats and humans. Serum corticosterone and hypothalamic paraventricular nucleus norepinephrine levels are decreased by Ad-36 infection in rats. Energy metabolism is altered because body weight and body fat increased in experimentally infected animals without increases in food intake. Some cytokines in adipocytes in vitro are increased and others are decreased including interleukins, leptin, and adiponectin. In tissue culture, differentiation of preadipocytes and accumulation of intracellular lipids is enhanced in both mouse and human stem cells.

The mechanisms responsible for these changes are changes in gene expression or protein levels of multiple enzymes and transcription factors. Ad-36 infection alters lipid and glucose metabolism, gene expression, and body composition. The Ad-36 viral E4orfl gene directly affects lipogenic enzymes to produce adiposity. Specifically, the E4orfl gene stimulates lipogenic enzymes in adipocytes, including sterol regulatory element binding protein (SREBP-1) and fatty acid synthase (FAS), and is associated with increased number of adipocytes and increased fat accumulation within adipocytes in vitro and in vivo. Both native Ad-36 and the E4orfl gene of Ad-36 cause malignant changes in cells in tissue culture including increasing cell growth, promoting migration, and altering enzymes or transcription factors in a manner consistent with malignancy.

Unsuspected natural infection has been found to occur in multiple experimental animal species. Indeed, twenty-five percent of rats purchased from a national experimental animal supply vendor were found to be infected on arrival at the University of Wisconsin. These experimental animals were fed high levels of dietary fat and it was discovered that infected rats gained about four times as much weight as uninfected rats. Mice obtained from the Virginia Commonwealth University vivarium were evaluated by quantitative PCR on adipose tissue. Of 140 mice tested for adenovirus infection, 39% were positive for Ad-36 DNA in adipose tissue. When mice were experimentally infected with Ad-36, the mice underwent increases in weight and adipose tissue and have alterations of serum lipids.

In the Longitudinal Aging Study at the Wisconsin Regional Primate Research Center, rhesus monkeys were studied every six months over seven years. All 15 monkeys became infected over the course of the study, presumably from human lab workers or from material from infected animals carried into their animal room by a lab worker. With the onset of infection the monkeys gained weight and had an unexpected decrease in blood cholesterol levels.

Fifty percent of rabbits used for antibody production were found to already have Ad-36 antibodies and 66% of lots of calf serum used in tissue culture experiments contained Ad-36 antibodies. The presence of antibodies in these experimental animals had major effects on the outcomes of the experiments, including interfering with serum neutralization assays by producing positive assays, blocking virus effects on tissue culture experiments to identify antiviral agents thus invalidating them, and blocking virus effects on tests of malignant potential of Ad-36 in, for example, breast cells that become malignant with Ad-36.

Accordingly, to avoid inaccurate results, it is important to screen experimental animals to determine if lipogenic adenovirus infection is present. If the experimental animals are determined to be positive for lipogenic adenovirus infection, then corrective measures may be taken. Corrective measures may include preventive measures, disinfecting or sterilization of equipment, vivariums, or cages, isolation of infected animals, treatment of infected animals, administering an antiviral agent (e.g., in a sufficient amount to significantly reduce or eliminate the adenovirus infection), notifying the researcher that the animal is positive for lipogenic adenovirus infection, and/or emergency slaughter of infected animals. If animals are not positive for lipogenic adenovirus infection, then preventive measures may be taken to ensure that these animals do not later become infected. Specifically, for example, the uninfected experimental animals may be administered a lipogenic adenovirus vaccine to prevent infection, as described in more detail below.

The experimental animals may be assessed for lipogenic adenovirus infection by the animal supplier or by the researcher(s) prior to conducting any experiments, and in particular, prior to conducting any experiments directed to fat or glucose metabolism, energy metabolism, and obesity research. If the researcher determines that the experimental animal is positive for lipogenic adenovirus infection, then the researcher may consider not using that particular animal, or may consider taking corrective actions or measures. Corrective actions or measures may include without limitation, procedures to be followed when as a result of a test or assay such as disinfection of cages and/or animal houses, emergency slaughter, preventive or remedial measures, administration of antiviral agents, and/or notifying a competent authority.

The prevalence of natural infection of lipogenic adenovirus, such as adenovirus type 36 (Ad-36), in experimental animals, food animals, and domestic animals in shown in Table 1. Given the trend of natural infection of lipogenic adenovirus type 36 (Ad-36) in a variety of animals, it is believed that Ad-36 natural infection also occurs in cattle, pigs, and sheep, just to name a few additional species.

| Animal | Number/ | Number/ | Number/ | Total | |
|---|---|---|---|---|---|
| Species | % Positive | % Negative | % Equivocal | Number | Category |
| Mice | 30/19% | 97/62% | 30/19% | 157 | Lab |
| Rats | 12/26% | 34/74% | 0% | 46 | Lab |
| Rabbits | 4/50% | 4/50% | 0% | 8 | Lab |
| Dogs | 3/75% | 1/25% | 0% | 4 | Lab |
| Cows | 4/40% | 6/60% | 0% | 10 | Lab |
| Horses | 2/100% | 0% | 0% | 2 | Lab |
| Monkeys | 16/100% | 0% | 0% | 16 | Lab |
| Chickens | 36/12% | 236/81% | 19/7% | 291 | Food |
| Cats | 8/40% | 10/50% | 2/10% | 20 | Domestic |
| Dogs | 14/93% | 0% | 1/7% | 15 | Domestic |
| Horses | 216/47% | 228/50% | 12/3% | 456 | Domestic |

Embodiments of the invention include the detection of a lipogenic adenovirus alone or in combination with the detection of specific biomarkers that are selectively expressed with lipogenic adenovirus infection. The lipogenic adenovirus and biomarkers may be detected by any method known in the art, including, but not limited to nucleic acid and/or protein detection techniques, PCR, and antibody-based methods (including but not limited to immuno-cytochemistry).

Lipogenic adenoviruses may include adenoviruses that are capable of stimulating increase lipid production in cells, tissues, and/or organs by facilitating expression of lipogenic enzymes, which in turn produce excess fatty acids and promote fat storage. The lipogenic adenoviruses of the invention may include Ad-5, Ad-36, and Ad-37, for example.

While not intending to be limited to a particular mechanism, in some embodiments, the molecular mechanism of lipogenic adenovirus infection is the stimulation of lipogenic enzymes that increase fat deposition in adipose tissues and cause differentiation of adult stem cells in adipose tissue to adipocytes. Specific lipogenic enzymes may be over-expressed or expressed in the cell, such as fatty acid synthase (FAS), glycerol-3-phosphodehydrogenase, lipoprotein lipase (LPL), SREBP-1, SCD1, CPT 1, PPAR-gamma, and L-type pyruvate kinase. In addition, Glut 1 and Glut 4 gene expression and PI3K expression may be increased in the cell in response to lipogenic adenovirus infection. These lipogenic enzymes may be responsible for the formation of excess fatty acids and promote fat storage within the cells of multiple organs.

### Biomarkers

A specific embodiment of the invention is directed to biomarkers that are characteristic of lipogenic adenovirus infection. The biomarkers of lipogenic adenovirus infection include lipogenic enzymes such as FAS, PPAR family proteins, C/EBP, ADD-1/SREBP-1, glycerol-3-phosphdehydrogenase, and lipoprotein lipase. Depending on the cell type, following lipogenic adenovirus infection, lipogenic enzymes may by expressed in cells not normally expressing the specific lipogenic enzyme(s) or over-expressed in cells that normally express the specific lipogenic enzyme(s).

For example, FAS is normally expressed in adult cells such as epithelial cells of the duodenum and stomach, hemopoietic cells, appendix, ganglion cells of alimentary tract, hepatocytes, mast cells, seminal vesicle, umbrella cells of urinary bladder, adrenal zona fasciculate cells, adipocytes, anterior pituitary cells, basket cells of cerebellum, cerebral cortical neurons, deciduas, decidualized stromal cells of endometrium, epithelial cells of apocrine gland, duct and acinus of breast, prostate, and sebaceous gland, letein cells, and Type II alveolar cells of lung. Additionally, FAS is also expressed in fetal cells such as anterior pituitary cells, chondrocytes of tracheobronchial wall, endothelium of blood vessels and heart, epithelial cells of bronchus, esphagogastrointestinal tract, lung, pancreas, prostate, thyroid, tongue, trachea, proximal tubules of kidney, fibroblasts, nodal lymphocytes, neuroblasts in adrenal medulla, thymocytes, striated myocytes of tongue, epithelial cells of salivary glands and tracheobronchial glands, hemopoietic cells, heptocytes, Lanhans cells of chorionic villi, osteoblasts, perivertebral fibroblastic cells, Schwann cells of sympathetic ganglion and Auerbach plexus, subcapsular cells of adrenal, adipocytes, Leidig cells of testis, mast cells, uroepithelium of urinary tract, and adrenocortical cells of upper layer. Therefore, a baseline value of FAS expression can be determined in these cells and then compared with an over-expression value in cells infected with lipogenic adenovirus.

### Immunoanalytical Detection Methods

Antibodies reactive to lipogenic adenoviruses and biomarker proteins may be employed for the detection of lipogenic adenovirus and biomarker proteins in a subject. Exemplary screening immunoanalytical techniques include without limitation, standard virus neutralization assay techniques or enzyme immunoassay techniques well known in the art. The standard virus neutralization assay may be used to identify the presence of antibodies reactive to a lipogenic adenovirus in a biological sample such as a serum sample obtained from a subject. A standard virus neutralization assay is described in the specific examples, below.

Techniques for raising and purifying antibodies against these lipogenic adenoviruses or fragments thereof (e.g., fiber protein, hexon protein, or fragments thereof), or other proteins (or fragments thereof) from these viruses for use in these immunoassay techniques may be prepared by conventional techniques are well known in the art. In a specific embodiment of the invention, antibodies will bind lipogenic adenovirus virus or lipogenic adenovirus proteins from solution as well as react with these proteins on Western or immunoblots or polyacrylamide gels. In a specific embodiment, the following fiber protein sequences, in Table 2 below, may be employed to generate antibodies reactive to lipogenic adenoviruses or may be used to detect lipogenic adenovirus antibodies (e.g., in a serum neutralization assay):

TABLE 2

| Amino Acid | Amino Acid End | |
|---|---|---|
| Start Position | Position | Sequence |
| 11 | 17 | FNPVYPY (SEQ ID NO.: 1) |
| 24 | 35 | NIPFLTPPFVSS (SEQ ID NO.: 2) |
| 41 | 55 | FPPGVLSLKLADPIA (SEQ ID NO.: 3) |
| 57 | 73 | ANGNVSLKVGGGLTVEQ (SEQ ID NO.: 4) |
| 75 | 88 | SGKLSVDTKAPLQV (SEQ ID NO.: 5) |
| 113 | 121 | AGHGLAVVT (SEQ ID NO.: 6) |
| 126 | 138 | SLPSLVGTLVVLT (SEQ ID NO.: 7) |
| 189 | 195 | PSPNCKV (SEQ ID NO.: 8) |
| 201 | 232 | SKLTLALTKCGSQILATVSLLVVTGKYAIISD (SEQ ID NO.: 9) |
| 235 | 254 | NPKQFSIKLLFNDKGVLLSD (SEQ ID NO.: 10) |
| 275 | 281 | YKEAVGF (SEQ ID NO.: 11) |
| 316 | 329 | LGGEVYQPGFIVVK (SEQ ID NO.: 12) |
| 336 | 342 | ANCAYSI (SEQ ID NO.: 13) |
| 348 | 359 | WGKVYKDPIPYD (SEQ ID NO.: 14) |
| | | VETARDSKLT (SEQ ID NO.: 15) |
| | | LGGEVYQPGFIVVK (SEQ ID NO.: 16) |
| | | WGKVYKDPIPYD (SEQ ID NO.: 17) |
| | | GTGSSAHG (SEQ ID NO.: 18) |

In another specific embodiment, antibodies will detect the presence of lipogenic adenovirus or lipogenic adenovirus proteins in a biological sample, using immunocytochemical techniques. Specific embodiments relating to methods for detecting lipogenic adenovirus or lipogenic adenovirus proteins include methods well known in the art such as enzyme linked immunosorbent assays (ELISA), radioimmunoassay (RIA), immunoradiometric assays (IRMA) and immunoenzymatic assays (IFMA), including sandwich assays using monoclonal and/or polyclonal antibodies.

### Nucleic Acid Detection Methods

The lipogenic adenoviruses and biomarkers may be detected by nucleic acid detection techniques, such as PCR and non-PCR techniques. The nucleic acid probe hybridization assay techniques used in these methods of the invention will be standard techniques (optionally after amplification of DNA or RNA extracted from a sample of blood, other body fluid, feces, tissue or organ) using nucleic acid probes (and primers if amplification is employed) made available by the lipogenic adenoviruses identified and made available by the invention. The sequences of nucleic acids characteristic of the lipogenic adenoviruses can be determined by standard techniques once the viruses are conventionally isolated, and probes and primers that are specific for the viruses and that provide the basis for nucleic acid probes and primers that can be used in nucleic acid based assays for the viruses are prepared using conventional techniques on the basis of the sequences.

Initially, screening involves amplification of the relevant lipogenic adenovirus sequences. In a specific embodiment of the invention, the screening method involves a non-PCR based strategy. Such screening methods include two-step label amplification methodologies that are well known in the art. Both PCR and non-PCR based screening strategies can detect target sequences with a high level of sensitivity.

One embodiment of the invention relates to target amplification. Here, the target nucleic acid sequence is amplified with polymerase. One specific method using polymerase-driven amplification is the polymerase chain reaction (PCR). The polymerase chain reaction and other polymerase-driven amplification assays can achieve over a million-fold increase in copy number through the use of polymerase-driven amplification cycles. Once amplified, the resulting nucleic acid can be sequenced or used as a substrate for DNA probes.

Quantitative amplification methods (e.g., quantitative PCR or quantitative linear amplification) can be used to quantify the amount of target nucleic acids. Methods of quantitative amplification are disclosed in, e.g., U.S. Pat. Nos. 6,180,349; 6,033,854; and 5,972,602, as well as in, e.g., Gibson et al., Genome Research 6:995-1001 (1996); DeGraves, et al., Biotechniques 34(1):106-10, 112-5 (2003); Deiman B, et al., Mol. Biotechnol. 20(2):163-79 (2002). Amplifications may be monitored in “real time.”

In general, quantitative amplification is based on the monitoring of the signal (e.g., fluorescence of a probe) representing copies of the template in cycles of an amplification (e.g., PCR) reaction. In the initial cycles of the PCR, a very low signal is observed because the quantity of the amplicon formed does not support a measurable signal output from the assay. After the initial cycles, as the amount of formed amplicon increases, the signal intensity increases to a measurable level and reaches a plateau in later cycles when the PCR enters into a non-logarithmic phase. Through a plot of the signal intensity versus the cycle number, the specific cycle at which a measurable signal is obtained from the PCR reaction can be deduced and used to back-calculate the quantity of the target before the start of the PCR. The number of the specific cycles that is determined by this method is typically referred to as the cycle threshold (Ct). Exemplary methods are described in, e.g., Heid et al. Genome Methods 6:986-94 (1996) with reference to hydrolysis probes.

One method for detection of amplification products is the 5′-3′ exonuclease “hydrolysis” PCR assay (also referred to as the TaqMan™ assay) (U.S. Pat. Nos. 5,210,015 and 5,487,972; Holland et al., Proc. Natl. Acad. Sci. USA 88: 7276-7280 (1991); Lee et al., Nucleic Acids Res. 21: 3761-3766 (1993)). This assay detects the accumulation of a specific PCR product by hybridization and cleavage of a doubly labeled fluorogenic probe (the “TaqMan™” probe) during the amplification reaction. The fluorogenic probe consists of an oligonucleotide labeled with both a fluorescent reporter dye and a quencher dye. During PCR, this probe is cleaved by the 5′-exonuclease activity of DNA polymerase if, and only if, it hybridizes to the segment being amplified. Cleavage of the probe generates an increase in the fluorescence intensity of the reporter dye.

Another method of detecting amplification products that relies on the use of energy transfer is the “beacon probe” method described by Tyagi and Kramer (Nature Biotech. 14:303-309 (1996)), which is also the subject of U.S. Pat. Nos. 5,119,801 and 5,312,728. This method employs oligonucleotide hybridization probes that can form hairpin structures. On one end of the hybridization probe (either the 5′ or 3′ end), there is a donor fluorophore, and on the other end, an acceptor moiety. In the case of the Tyagi and Kramer method, this acceptor moiety is a quencher, that is, the acceptor absorbs energy released by the donor, but then does not itself fluoresce. Thus, when the beacon is in the open conformation, the fluorescence of the donor fluorophore is detectable, whereas when the beacon is in the hairpin (closed) conformation, the fluorescence of the donor fluorophore is quenched. When employed in PCR, the molecular beacon probe, which hybridizes to one of the strands of the PCR product, is in the open conformation and fluorescence is detected, and the probes that remain unhybridized will not fluoresce (Tyagi and Kramer, Nature Biotechnol. 14: 303-306 (1996)). As a result, the amount of fluorescence will increase as the amount of PCR product increases, and thus may be used as a measure of the progress of the PCR. Those of skill in the art will recognize that other methods of quantitative amplification are also available.

Various other techniques for performing quantitative amplification of a nucleic acid are also known. For example, some methodologies employ one or more probe oligonucleotides that are structured such that a change in fluorescence is generated when the oligonucleotide(s) is hybridized to a target nucleic acid. For example, one such method involves a dual fluorophore approach that exploits fluorescence resonance energy transfer (FRET), e.g., LightCycler™ hybridization probes, where two oligo probes anneal to the amplicon. The oligonucleotides are designed to hybridize in a head-to-tail orientation with the fluorophores separated at a distance that is compatible with efficient energy transfer. Other examples of labeled oligonucleotides that are structured to emit a signal when bound to a nucleic acid or incorporated into an extension product include: Scorpions™ probes (e.g., Whitcombe et al., Nature Biotechnology 17:804-807, 1999, and U.S. Pat. No. 6,326,145), Sunrise™ (or Amplifluor™) probes (e.g., Nazarenko et al., Nuc. Acids Res. 25:2516-2521, 1997, and U.S. Pat. No. 6,117,635), and probes that form a secondary structure that results in reduced signal without a quencher and that emits increased signal when hybridized to a target (e.g., Lux Probes™)

In other embodiments, intercalating agents that produce a signal when intercalated in double stranded DNA may be used. Exemplary agents include SYBR GREEN™ and SYBR GOLD™. Since these agents are not template-specific, it is assumed that the signal is generated based on template-specific amplification. This can be confirmed by monitoring signal as a function of temperature because melting point of template sequences will generally be much higher than, for example, primer-dimers, etc.

In a specific embodiment, the following primers may be employed for PCR amplification of the nucleic acid sequence encoding the Ad-36 hexon protein:

SEQ ID NO.: 19:
5′-ggtggacaaccacccactac-3′ (Forward primer)
SEQ ID NO.: 20:
5′-tggacaaccacccactacaa-3′ (Forward primer)
SEQ ID NO.: 21:
5′-cagcggagcttgtatcttcc-3′ (Reverse primer)

In a another specific embodiment, nested PCR may be used to detect Ad-36 DNA in biological samples. Four primers were designed to unique regions of the Ad-36 fiber protein gene for use in a nested PCR assay for detection of viral DNA, which are as follows:

outer forward primer
(SEQ ID No.: 22)
(5′-gtctggaaaactgagtgtggata),
outer reverse primer
(SEQ ID No.: 23)
(5′-atccaaaatcaaatgtaatagagt),
inner forward primer
(SEQ ID No.: 24)
(5′-ttaactggaaaaggaataggta),
inner reverse primer
(SEQ ID No.: 25)
(5′-ggtgttgttggttggcttaggata).

In a specific embodiment, the following primers may be employed for detecting Ad-36 fiber protein using SYBRgreen method:

(SEQ ID NO.: 32)
Forward: 5′-taagccaaccaacaacacca-3′
(SEQ ID NO.: 33)
Reverse: 5′-tgcacaattggcatcagttt-3′
(SEQ ID NO.: 34)
Forward: 5′-ggccatggtttagcagttgt-3′
(SEQ ID NO.: 35)
Reverse: 5′-gtccaaagggtgcgtgtatc-3′
(SEQ ID NO.: 36)
Forward: 5′-ttaactggaaaaggaataggta-3′
(SEQ ID NO.: 37)
Reverse: 5′-ggtgttgttggttggcttaggata-3′

In another specific embodiment, the following primers may be employed for detecting Ad-36 fiber protein using the Taqman method:

Forward:
(SEQ ID NO.: 38)
5′-caatgggaatgtctcactcaaggt-3′
Reverse:
(SEQ ID NO.: 39)
5′-agggtgccttagtatccacact-3′
(SEQ ID NO.: 40)
5′-cagttttccagactgttgttct-3′

When the probes are used to detect the presence of the lipogenic adenovirus nucleic acid sequences or biomarker nucleic acid sequences, nucleic acid may be first isolated from the biological sample. The sample nucleic acid may be prepared in various ways known in the art, to facilitate detection of the target sequence, e.g., denaturation, restriction digestion, electrophoresis or dot blotting. The targeted region of the analyte nucleic acid usually must be at least partially single-stranded to form hybrids with the targeting sequence of the probe. If the sequence is naturally single-stranded, denaturation will not be required. However, if the sequence is double-stranded, the sequence will probably need to be denatured. Denaturation can be carried out by various techniques well known in the art.

Analyte nucleic acid and probe are incubated under conditions which promote stable hybrid formation of the target sequence in the analyte. The region of the probes which is used to bind to the analyte can be made completely complementary to the targeted region of the lipogenic adenovirus of interest, and in particular the Ad-36 fiber coat protein or hexon protein. For example, the following probes may be used to detect the nucleic acid encoding Ad-36 fiber coat protein:

SEQ ID NO: 26: 5′-agttgaaacagcaagagactcaaag-3′
SEQ ID NO: 27: 5′-ggtactggatcaagtgcacatggag-3′
SEQ ID NO: 28: 5′-ttgaaacagcaagagactcaaagctaac-3′

High stringency conditions may be desirable in order to prevent false positives. However, conditions of high stringency are used only if the probes are complementary to regions of the lipogenic adenovirus. The stringency of hybridization is determined by a number of factors during hybridization and during the washing procedure, including temperature, ionic strength, base composition, probe length, and concentration of formamide.

Detection, if any, of the resulting hybrid is usually accomplished by the use of labeled probes. Alternatively, however, the probe may be unlabeled, but may be detectable by specific binding with a ligand which is labeled, either directly or indirectly. Suitable labels, and method for labeling probes and ligands are well known in the art, and include, for example, radioactive labels which may be incorporated by known methods (e.g., nick translation, random priming or kinasing), biotin, fluorescent groups, chemiluminescent groups (e.g., dioxetanes) enzymes, antibodies, gold nanoparticles and the like. Variations of this basic scheme are known in the art, and include those variations that facilitate separation of the hybrids to be detected from extraneous materials and/or that amplify the signal from the labeled moiety.

As noted above, non-PCR based screening assays are also contemplated by this invention. This procedure hybridizes a nucleic acid probe (or analog such as a methyl phosphonate backbone replacing the normal phosphodiester) to the low level DNA target. This probe may have an enzyme covalently linked to the probe, such that the covalent linkage does not interfere with the specificity of the hybridization. The enzyme-probe-conjugate-target nucleic acid complex can then be isolated away from the free probe conjugate and a substrate is added for enzyme detection. Enzymatic activity is observed as a change in color development or luminescent output resulting in about a 10³ to about a 10⁶ increase in sensitivity.

Two-step label amplification methodologies are known in the art. These assays work on the principle that a small ligand (such as digioxigenin, biotin, or the like) is attached to a nucleic acid probe capable of specific binding the adenovirus sequence region of interest. In one example, the small ligand attached to the nucleic acid probe is specifically recognized by an antibody-enzyme conjugate. In one embodiment of this example, digioexigenin is attached to the nucleic acid probe. Hybridization is detected by an antibody-alkaline phosphatase conjugate which turns over a chemiluminescent substrate. In a second example, the small ligand is recognized by a second ligand-enzyme conjugate that is capable of specifically complexing to the first ligand. A well known embodiment of this example is the biotin-avidin type interactions.

It is also contemplated within embodiments that the nucleic acid probe assays of the invention will employ a cocktail of nucleic acid probes capable of detecting various species of adenoviruses. Thus, in one example to detect the presence of Ad-36, Ad-37 and/or Ad-5, for example, in a biological sample, more than one probe complementary of the targeted regions of interest in the various types of adenovirus may be employed.

As the skilled will understand, more than one strain of lipogenic adenovirus may be tested for simultaneously in an immunological or nucleic acid-based assay method for testing for virus in accordance with the invention and kits may be assembled to facilitate carrying out the methods for a particular virus or a plurality of them.

### Correlating

In making a correlation based on presence of lipogenic adenovirus biomarkers, such as the expression or over-expression of lipogenic enzymes, the presence of a lipogenic adenovirus biomarker may be compared to a threshold value that distinguished between one diagnosis, determination of a predisposition, risk assessment, etc., to another. The threshold value or range may be determined by measuring the presence of lipogenic adenovirus biomarker in diseased and normal samples using the particular desired assay and then determining a value that distinguished at least a majority of the abnormal adipose tissue hypertrophy from a majority of non-adipose tissue hypertrophy samples.

A manual comparison can be made or a computer can compare and analyze the values to detect disease, assess the risk of contracting disease, determining a predisposition to disease, stage disease, diagnose disease, monitor, or aid in the selection of treatment for a person with disease.

In some embodiments the threshold value is set such that there is at least 10, 20, 30, 40, 50, 60, 70, 80% or more sensitivity and at least 70% specificity with regard to detecting abnormal adipose tissue hypertrophy. In some embodiments, the methods comprise recording a diagnosis, prognosis, risk assessment or classification, based on lipogenic adenovirus status determined from an individual. Any type of recordation is contemplated, including electronic recordation, e.g., by a computer.

### Computer Program

The correlations for the methods described herein can involve computer-based calculations and tools. The tools are advantageously provided in the form of computer programs that are executable by a general purpose computer system (referred to herein as a “host computer”) of conventional design. The host computer may be configured with many different hardware components and can be made in many dimensions and styles (e.g., desktop PC, laptop, tablet PC, handheld computer, server, workstation, mainframe). Standard components, such as monitors, keyboards, disk drives, CD and/or DVD drives, and the like, may be included. Where the host computer is attached to a network, the connections may be provided via any suitable transport media (e.g., wired, optical, and/or wireless media) and any suitable communication protocol (e.g., TCP/IP); the host computer may include suitable networking hardware (e.g., modem, Ethernet card, WiFi card). The host computer may implement any of a variety of operating systems, including UNIX, Linux, Microsoft Windows, MacOS, or any other operating system.

Computer code for implementing aspects of the invention may be written in a variety of languages, including PERL, C, C++, Java, JavaScript, VBScript, AWK, or any other scripting or programming language that can be executed on the host computer or that can be compiled to execute on the host computer. Code may also be written or distributed in low level languages such as assembler languages or machine languages.

The host computer system advantageously provides an interface via which the user controls operation of the tools. In the examples described herein, software tools are implemented as scripts (e.g., using PERL), execution of which can be initiated by a user from a standard command line interface of an operating system such as Linux or UNIX. Those skilled in the art will appreciate that commands can be adapted to the operating system as appropriate. In other embodiments, a graphical user interface may be provided, allowing the user to control operations using a pointing device. Thus, the present invention is not limited to any particular user interface.

Scripts or programs incorporating various features of the invention may be encoded on various computer readable media for storage and/or transmission. Examples of suitable media include magnetic disk or tape, optical storage media such as compact disk (CD) or DVD (digital versatile disk), flash memory, and carrier signals adapted for transmission via wired, optical, and/or wireless networks conforming to a variety of protocols, including the Internet.

### Treatments

Subsequent to a determination of whether an animal has been infected with a lipogenic adenovirus, the animal may need specialized treatment. For example, animals who are negative for lipogenic adenovirus may be prescribed vaccines or other agents that will prevent infection.

The vaccines of the invention are effective to prevent infection of lipogenic adenovirus in an animal and may be made by conventional methods known in the art. In some embodiments, the vaccines include an immunogenic component that is live, inactive virus, killed virus, antigenic peptide generated from the fiber coat protein or hexon protein, or an epitope-comprising segment thereof. In some embodiments, an anti-lipogenic adenovirus vaccine of the invention, where the active ingredient is nucleic acid, will also be a standard preparation for vaccines of that type. With vaccines of this type, the nucleic acid is not the immunogen but is expressed in vivo after administration of the vaccine as a peptide or protein which in turn is immunogenic. Vaccines of this type will be administered by techniques known in the art for such vaccines (e.g., intramuscular injection). Dosing will also be according to procedures known in the art to cause and maintain protective immunity against lipogenic adenovirus infection in the vaccinated animal.

The vaccine compositions may include carriers, excipients, adjuvants, buffers, antimicrobials, preservatives and the like as well understood in the art. Thus, in addition to the active ingredient, in some embodiments, the vaccines will have suitable compositions, usually aqueous buffers, such as phosphate-buffered saline or the like, in which the active ingredient will be suspended along with, optionally, any of various immune-system stimulating adjuvants used in animal vaccine preparations, antimicrobial compositions, and other compositions to stabilize the preparations. All compositions included with the vaccine preparation will be suitable for administration to animals. The vaccine preparation may be stored in lyophilized form and then combined with solution soon before administration. For oral administration, the vaccine composition may be in solution, tablet or pill form. The concentration of active component in solution with which it is administered typically will be between about 1 ng and about 1 mg/ml. The anti-lipogenic adenovirus vaccines of the invention will be administered intranasally, orally, or by injection intravenously, intramuscularly, subcutaneously or peritoneally.

Appropriate dosing of the lipogenic adenovirus vaccine is well within the skill of medical practitioners and will depend on a number of factors including the age of the animal being treated, the urgency of the animal's developing protective immunity, the status of the animal's immune system, and other factors known to those of skill in the art. The vaccine typically will be administered in several steps in order to cause and maintain protective immunity against lipogenic adenovirus in the animal being vaccinated. Thus, after the primary vaccination, there may be between one and about ten booster vaccinations separated by periods between about 1 week and 10 years.

The anti-adipogenic adenovirus vaccine may include an effective dose of an active ingredient such as a killed adenovirus type 36, an inactivated adenovirus type 36, a protein or peptide sequence encoding an adenovirus 36 coat protein or fragment thereof (e.g., at least 10, 15, 20, 30, 50, or more amino acids), an nucleic acid sequence encoding an adenovirus type 36 coat protein or a fragment thereof, an adenovirus type 36 E1A protein, a genetically modified non-pathogenic virus, and a non-pathogenic virus.

In some embodiments, the anti-adipogenic adenovirus vaccines (including but not limited to aspects in which the immunogenic component is live, inactivated virus, killed virus, coat protein per se, epitope-comprising coat protein segment, or coat protein (or epitope-comprising segment thereof)) can be deliver as part of, or encoded by, a non-pathogenic, genetically modified carrier virus such as a vaccinia virus or a fowl pox virus, are prepared using methods well known in the art. Thus, in some embodiments, the vaccines will include carriers, excipients, adjuvants, antimicrobials, preservatives and the like as well understood in the art. Thus, in addition to the active ingredient, the vaccines will have suitable compositions, usually aqueous buffers, such as phosphate-buffered saline or the like, in which the active ingredient will be suspended along with, optionally, any of various immune-system stimulating adjuvants used in vaccine preparations, antimicrobial compositions, and other compositions to stabilize the preparations. All compositions included with the vaccine preparation will be suitable for administration. In some embodiment, the vaccine preparation may be stored in lyophilized form and then combined with solution soon before administration. For oral administration, the vaccine preparation may be in solution, tablet or pill form optionally with an enteric coating as understood in the art. The concentration of active (immunogenic or immunogen-providing) component in solution with which it is administered typically will be between about 1 ng and about 1 mg ml.

In some embodiments, a single dose of an anti-obesity vaccine (in solution foam) will have a volume of between about 0.1 ml and 10 ml and, in any form, will have between about 1 ng and 10 mg of killed or inactivated adipogenic virus, between about 1 ng and 10 mg of genetically modified, non-pathogenic virus, or between about 1 ng and 10 mg of coat protein (e.g., fiber protein) or 6-30 amino acid peptide (in its form as modified to be immunogenic).

In other embodiment, an anti-adipogenic adenovirus vaccine, wherein the active ingredient is nucleic acid, will also be a standard preparation for vaccines of that type. With vaccines of this type, the nucleic acid is not the immunogen but is expressed in vivo after administration of the vaccine as a peptide or protein which in turn is immunogenic. Vaccines of this type will be administered by techniques known in the art for such vaccines (e.g., intramuscular injection). Dosing will also be according to procedures known in the art to cause and maintain protective immunity against viral obesity in the vaccinated animal.

Note that an anti-adipogenic adenovirus vaccine according to the invention may include active ingredients based on more than one adipogenic virus (or the coat protein (e.g. fiber protein) or epitopic segments of the coat protein thereof).

Without further elaboration, it is believed that one skilled in the art using the preceding description can utilize the invention to the fullest extent. The following examples are illustrative only, and not limiting of the disclosure in any way whatsoever.

### EXAMPLES

### Specific Example 1

### Ad-36 Infection in Domestic Animals and Animals Suitable for Consumption

The study set forth in this example determined the prevalence of unsuspected (natural) Ad-36 infection in domestic animals.

61 pieces of packaged chicken were obtained from local grocery stores in Virginia and tested for lipogenic adenovirus infection by quantitative PCR. 4 of 10 lots of calf serum or fetal bovine serum used for tissue culture media were assayed for the presence of Ad-36 antibodies. Stored serum from horses was assayed in duplicate by serum neutralization assay (SN) at the Obetech laboratory. A titer of 1:8 in both duplicates was considered positive for Ad-36 antibodies. Serum from 21 cats from veterinary clinics at Ohio State and Mechanicsville Animal Hospital (MAH) were assayed by SN. Adipose tissue obtained from nine dogs at MAH and VA Commonwealth Univ. was assayed by real time polymerase chain reaction assay (qPCR) using specific TaqMan^{R} primers and probe on an ABI StepOne^{R} Plus qPCR apparatus.

9 of the 61 pieces of packages of packaged chicken were positive for Ad-36 (15%). There were 287 usable serum neutralization assays in horses. Ad-36 antibodies were found at a titer of 1:8 or above in both duplicates in 137 horses (48%) and negative in 104 (36%). Equivocal results, a titer of 1:8 in one replicate and <1:8 in the other, were obtained in 45 horses (16%). Twelve of the 21 cats were positive for Ad-36 (57%). Fifteen of the 17 tested by serum neutralization were positive (88%). Seven of nine dogs (78%) were positive for viral DNA in adipose tissue. Four positive animals at MAH were obese and two negative animals were not obese by a blinded rating on a 1-10 scale. 4 of 10 lots of calf or fetal bovine serum all of which were used for media for tissue culture had Ad-36 antibodies. The presence of Ad-36 antibodies in calf or fetal bovine serum provides strong evidence that cows are Ad-36 positive.

Additionally, adipose tissue from three chicken pieces that tested positive for Ad-36 DNA by PCR were homogenized, centrifuged, and the supernatant placed on A549 cells in tissue culture. A virus from all three chicken fat samples survived and grew in the A549 cells. PCR analysis showed that the virus was Ad-36. This is evidence that chicken sold in supermarkets contains live Ad-36 virus.

Summary: These results demonstrate that 50% or more of domestic animals were positive for Ad-36 infection by either SN or PCR.

### Specific Example 2

A virus neutralization assay (serum neutralization assay) was used to assay serum for antibody reactive with lipogenic adenovirus in serum of test subjects. First, serum was thawed and heat-inactivated for about 30 minutes at 56° C. The assay was carried out in standard 96-well microtiter plates. Serial two fold dilutions (1:2 to 1:1024) were made with the medium that is the A549 growth medium described in Example 3 but lacks the fetal calf serum and sodium bicarbonate. 50 microliters of each dilution was added in duplicate to the wells of the plate. 50 microliters of virus suspension (100 TCID₅₀) was then added to each well. (TCID₅₀ was calculated by serially diluting viral stock solution and inoculating A549 cells with the dilutions to determine the reciprocal of the highest dilution of virus which causes CPE in 50% of the material inoculated.) The plates were then incubated at 37° C. for 1 hour. Then 100 microliters of A549 cell suspension, containing approximately 20,000 cells, was added to each well and the plate was further incubated at 37° C. for 12 days. Crystal violet-ethanol was then added to each of the wells to fix and stain the cells and the plates were examined macroscopically for CPE. The highest serum dilution with no CPE is the titer. Controls used in the procedure were wells with no virus and wells with virus but no serum. A back titration was carried out to confirm that appropriate virus dilutions were used. Positive control was antisera to chicken adenovirus and human adenovirus. Presence of CPE with the virus and no CPE in the presence of serum was considered an indication of effective neutralization of the virus with antibody in serum, such that the serum was considered to have antibody against the virus. A titer of 1:8 or greater was considered positive.

### Specific Example 3

Serum from 40 rats bought from Harlan Labs (IN), 15 rhesus monkeys from the Aging Study, Wisconsin National Primate Center, 8 rabbits from US Biological (MA), 20 cats from the Veterinary Teaching Hospital, Ohio State U., and 300 horses from Virginia Polytechnic Institute was obtained. 10 lots of calf serum and 2 lots of horse serum was obtained. Fat tissue from 140 mice and 4 dogs was obtained from the Virginia Commonwealth University vivarium.

Ad-36 status was assayed by serum neutralization assay (positive=titer>1:8). Rats were fed a 36% or 48% kcal from fat diet and weight gain and body fat by proximate analysis was measured.

### Results

25% of rats from Harlan Labs arrived with unsuspected Ad-36 infection. Hi fat feeding resulted in greater weight gains (36.6 versus 9.6 g, p<0.02) and 51% greater body fat (82 versus 54 g, p<00.02) in infected versus uninfected rats.

100% of monkeys became Ad-36 positive over the 7 year study and all monkeys gained weight and exhibited lowered serum cholesterol post infection (p<0.05). Blood was drawn every 6 months for 7 years. All 15 monkeys were infected at the end of 7 years.

39% of mice from the Virginia Commonwealth University vivarium were positive for Ad-36 DNA by quantitative PCR. Previous studies have shown that mice experimentally infected with Ad-36 increases body weight and body fat and have paradoxically reduced levels of serum lipid (e.g., cholesterol, LDL cholesterol, and triglycerides). 3 out of the 4 dogs were positive for Ad-36 infection, which was determined by qualitative PCR.

50% of rabbits (4/8) were Ad-36 positive on arrival. Rabbits were being tested for ability of Ad-36 component proteins to generate antibodies as candidates for a vaccine. Rabbits with antibodies at baseline in effect received a “booster” and were worthless for evaluation of the candidate vaccines. 59% of horses and 58% of cats were Ad-36 positive.

4 of 10 lots of calf or fetal bovine serum and 2 of 2 lots of horse serum, all of which were used for media for tissue culture had Ad-36 antibodies. The Ad-36 antibodies blocked the virus in serum neutralization assays, cancer biology experiments, and evaluations of candidate antiviral agents, causing these assays or experiments to be worthless.

Unsuspected infection with Ad-36 occurs in laboratory animals from all sources, including animal suppliers, in university vivariums, and in domestic animals in veterinary clinics. Ad-36 infection necessitated reanalysis of the data or simply discarding it. At a minimum, the variability of the outcome measures may be increased. The variety of ways in which outcome variables may be affected means that studies in many different areas may already have been compromised (e.g. diabetes, cardiovascular, and perhaps cancer research) by adenovirus infection. To prevent these problems in the future, it may be that animals being used for experimental testing, such as drug testing, should be tested for and/or vaccinated against lipopgenic adenovirus infection such as Ad-36, prior to use in experiments. Governmental regulatory bodies such as the FDA, may wish to consider establishing rules and/or GLP/GMP for such experimental animals.

### Specific Example 4

The DNA sequence encoding Ad-36 (SEQ ID No.: 29) is as follows:

5′taacgaagaggctgtgaatttaatatttccagaatctatgattcttcaggctgacatagccagtgaagccatagttactcctctacatact
cccactctgcctcccatacctgaattggaggaggatgaagaaatagacctccggtgctacgaggaaggttttcctcccagcgattcaga
ggacgaacagggtgagcagcagatggctctaatctctgatttagcttgtgtgattgtggaggaacaagttgtgattgaaaaatctaccga
gccagtacaaggctgtaggaactgccagtatcaccgggataagtccggagacccgaacgcttcctgcgctctgtgttacatgaaatct
actttcagctttatttacagtaagtggagtgaatgtgagagaggctgagtgcttaacacataactgtaatgcttgaacagctgtgctaagtg
tggtttatttttgttactaggtccggtgtcagaggatgagtcatcaccctcagaagaagaccacccgtctccccctgagctgtcaggcga
aacgcccctgcaagtgcacagacccaccccagtcagagccagtggcgagaggcgagcagctgtagaaaaaattgaggacttgttac
atgacatgggtggggatgaacctttggacctgagcttgaaacgccccaggaactaggcgcagctgcgcttagtcatgtgtaaataaag
ttgtacaataaaagtatatgtgacgcatgcaaggtgtggtttatgactcatgggcggggcttagtcctatataagtggcaacacctgggc
acttgggcacagaccttcagggagttcctgatggatgtgtggactatccttgcagactttagcaagacacgccggcttgtagaggatag
ttcagacgggtgctccgggttctggagacactggtttggaactcctctatctcgcctggtgtatacagttaagaaggattataaagagga
atttgaaaatctttttgctgactgctctggtctgctagattctctgaatcttggccaccagtcccttttccaggaaagggtactccacagcctt
gatttttccagcccagggcgcactacagccggggttgcttttgtggtttttctggttgacaaatggagccaggacacccaactgagcag
gggctacatcctggacttcgcagccatgcacctgtggagggcctggatcaggcagcggggacagagaatcttgaactactggcttct
acagccagcagctccgggtcttcttcgtctacacagacaaacatccatgttggaggaagaaatgaggcaggccatggacgagaaccc
gaggagcggcctggaccctccgtcggaagaggagctggattgaatcaggtatccagcctgtacccagagcttagcaaggtgctgac
atccatggccaggggagtgaagagggagaggagcgatgggggtaataccgggatgatgaccgagctgactgccagtctgatgaat
cggaagcgcccagagcgccttacctggtacgagctacagcaggagtgcagggatgagataggcctgatgcaggataaatatggcct
ggagcagataaaaacccattggttgaacccagatgaggattgggaggaggctattaagaagtatgccaagatagccctgcgcccaga
ttgcaagtacatagtgaccaagaccgtgaatatcagacatgcctgctacatctcggggaacggggcagaggtggtcatcgataccctg
gacaaggccgccttcaggtgttgcatgatgggaatgagagcaggagtgatgaatatgaattccatgatcttcatgaacattaagttcaat
ggagagaagtttaatggggtgctgttcatggccaacagccacatgaccctgcatggctgcagcttcttcggtttcaacaacatgtgcgc
cgaggtctggggagctgctaagatcaggggatgtaagttttatggctgctggatgggcgtggtcggaagacccaagagcgagatgtc
tgtgaagcagtgtgtgtttgagaaatgctacctgggagtctctaccgagggcaatgctagagtgagacactgctcttccatggagacgg
gctgcttctgcctggtgaagggcacggcctctctgaagcataatatggtgaagggctgcacggatgagcgcatgtacaacatgctgac
ctgcgattcgggggtctgccatatcctgaagaacatccatgtgacctcccaccccagaaagaagtggccagtgtttgagaataacctgc
tgatcaagtgccatatgcacctgggtgccagaaggggcaccttccagccgtaccagtgcaactttagccagaccaagctgctgttgga
gaacgatgccttctccagggtgaacctgaacggcatctttgacatggatgtctcggtgtacaagatcctgagatacgatgagaccaagt
ccagggtgcgcgcttgcgagtgcgggggcagacacaccaggatgcaaccagtggccctggatgtgacagaggagctgagaccag
accacctggtgatggcctgtaccgggaccgagttcagctccagtggggaggacacagattagaggtaggttttgagtagtgggcgtg
gctaaggtgagtataaaggcggtgtcttacgagggtctttttgcttttctgcagacatcatgaacgggaccggcggggccttcgaaggg
gggctttttagcccttatttgacaacccgcctgccgggatgggccggagttcgtcagaatgtgatgggatctacggtggatgggcgccc
agtgcttccagcaaattcctcgaccatgacctacgcgaccgtggggagctcgtcgctcgacagcaccgccgcagccgcggcagccg
cagccgccatgacagcgacgagactggcctcgagctacatgcccagcagcagcagtagcccytctgtgcccagttccatcatcgcc
gaggagaaactgctggccctgctggcagagctggaagccctgagccgccagctggccgccctgacccagcaggtgtccgagctcc
gcgagcaacagcagcagcaaaataaatgattcaataaacacagattctgattcaaacagcaaagcatctttattatttattttttcgcgcgc
ggtaggccctggtccacctctcccgatcattgagagtgcggtggattttttccaggacccggtagaggtgggattggatgttgaggtac
atgggcatgagcccgtcccgggggtggaggtagcaccactgcatggcctcgtgctctggggtcgtgttgtagatgatccagtcatagc
aggggcgctgggcgtggtgctggatgatgtccttaaggaggagactgatggccacggggagccccttggtgtaggtgttggcgaag
cggttgagctgggagggatgcatgcggggggagatgatgtgcagtttggcctggatcttgaggttggcaatgttgccgcccagatccc
gcctggggttcatgttgtgcaggaccaccaggacggtgtagcccgtgcacttggggaacttatcatgcaacttggaagggaatgcgtg
gaagaatttggagacgcccttgtgcccgcccaggttttccatgcactcatccatgatgatggcgatgggcccgtgggctgcggctttgg
caaagacgtttctggggtcagagacatcgtaattatgctcctgggtgagatcatcataagacattttaatgaatttggggcggagggtgc
cagattgggggacgatagttccctcgggccccggggcaaagttcccctcacagatctgcatctcccaggctttcatctcggaggggg
ggatcatgtccacctgcggggcgatgaaaaaaacggtttccggggcgggggtgatgagctgcgaggagagcaggtttctcaacagc
tgggacttgccgcacccggtcgggccgtagatgaccccgatgacgggttgcaggtggtagttcaaggacatgcagctgccgtcgtcc
cggaggaggggggccacctcgttgagcatgtctctgacttggaggttttcccggacgagctcgccgaggaggcggtccccgcccag
cgagaggagctcttgcagggaagcaaagtttttcaggggcttgagtccgtcggccatgggcatcttggcgagggtctgcgagaggag
ctccaggcggtcccagagctcggtgacgtgctctacggcatctcgatccagcagacttcctcgtttcgggggttgggacgactgcgac
tgtagggcacgagacgatgggcgtccagcgcggccagcgtcatgtccttccagggtctcagggtccgcgtgagtgtggtctccgtca
cggtgaaggggtgggccccgggctgggcgcttgcaagggtgcgcttgagactcatcctgctggtgctgaaacgggcacggtcttcg
ccctgcgcgtcggcgagatagcagttgaccatgagctcgtagttgagggcctcggcggcgtggcccttggcgcggagcttgcccttg
gaagagcgcccgcaggcgggacagagaagggattgcagggcgtagagcttgggtgcgagaaaaacggactcgggggcgaagg
cgtccgctccgcagtgggcgcagacggtctcgcactcgacgagccaggtgagctcgggctgctcggggtcaaaaaccagttttccc
ccgttctttttgatgcgcttcttacctcgcgtctccatgagtctgtgtccgcgctcggtgacaaacaggctgtcggtgtccccgtagacgg
acttgatgggcctgtcctgcagggacgtcccgcggtcctcctcgtagagaaactcggaccactctgagacaaaggcgcgcgtccacg
ccaagacaaaggaggccacgtgcgaggggtagcggtcgttgtccaccagggggtccaccttttccaccgtgtgcagacacatgtcc
ccctcctccgcatccaagaaggtgattggcttgtaggtgtaggccacgtgaccgggggtccccgacgggggggtataaaagggggc
gggtctgtgctcgtcctcactctcttccgcgtcgctgtccacgagcgccagctgttggggtaggtattccctctctagagcgggcatgac
ctcggcactcaggttgtcagtttctagaaacgaggaggatttgatgttggcctgccctgccgcgatgctttttaggagactttcatccatct
ggtcagaaaagacaatttttttattgtcaagcttggtggcaaaggagccatagagggcgttggatagaagcttggcgatggatctcatgg
tctgatttttgtcacggtcggcgcgctccttggccgcgatgtttagctggacatactcgcgcgcgacgcacttccattcggggaagacg
gtggtgcgctcgtcgggcacgatcctgacgcgccagccgcggttatgcagggtgaccaggtccacgctggtggccacctcgccgc
gcaggggctcgttggtccagcagagtctgccgcccttgcgcgagcagaacgggggcagcacatcaagcagatgctcgtcaggggg
gtccgcatcgatggtgaagatgcccggacagagttccttgtcaaaataatcgatttttgaggatgcatcatccaaggccatctgccactc
gcgggcggccagcgctcgctcgtaggggttgaggggcggaccccagggcatgggatgcgtgagggcggaggcgtacatgccgc
agatgtcatacacatagatgggctccgagaggatgccgatgtaggtgggataacagcgccccccgcggatgctggcgcgcacgtag
tcatacaactcgtgcgagggggccaagaaggcggggccgagattggtgcgctggggctgctcggcgcggaagacgatctggcga
aagatggcgtgcgagtttgaggagatggtgggccgttggaagatgttaaagtgggcgtgaggcaggcggaccgagtcgcggatga
agtgcgcgtaggagtcttgcagcttggcgacgagctcggcggtgacgaggacgtccatggcacagtagtccagcgtttcgcggatg
atgtcataacccgcctctcctttcttctcccacagctcgcggttgagggcgtactcctcgtcatccttccagtactcccggagcgggaatc
ctcgatcgtccgcacggtaagagcccagcatgtagaaatggttcacggccttgtagggacagcagcccttctccacggggagggcgt
aagcttgagcggccttgcggagcgaggtgtgcgtcagggcgaaggtgtctctgaccatgactttcaagaactggtacttgaaatccga
gtcgtcgcagccgccgtgctcccagagctcgaaatcggtgcgcttcttcgagagggggttaggcagagcgaaagtgacgtcattgaa
gagaatcttgcctgcccgcggcatgaaattgcgggtgatgcggaaagggcccgggacggaggctcggttgttgatgacctgggcgg
cgagcacgatctcgtcgaagccgttgatgttgtgcccgacgatgtagagttccatgaatcgcgggcggcctttgatgtgcggcagctttt
tgagctcctcgtaggtgaggtcctcggggcattgcagtccgtgctgctcgagcgcccactcctggagatgtgggttggcttgcatgaa
ggaagcccagagctcgcgggccatgagggtctggagctcgtcgcgaaagagacggaactgctggcccacggccatcttttcgggt
gtgacgcagtagaaggtgagggggtcccgctcccagcgatcccagcgtaagcgcacggcgagatcgcgagcgagggcgaccag
ctcggggtccccggagaatttcatgaccagcatgaaggggacgagctgcttgccgaaggaccccatccaggtgtaggtttctacatcg
taggtgacaaagagccgctccgtgcgaggatgagagccgattgggaagaactggatttcctgccaccagttggacgagtggctgttg
atgtgatgaaagtagaaatcccgccggcgaaccgagcactcgtgctgatgcttgtaaaagcgtccgcagtactcgcagcgctgcacg
ggctgtacctcatccacgagatacacagcgcgtcccttgaggaggaacttcaggagtggcggccctggctggtggttttcatgttcgcc
tgcgtgggactcaccctggggctcctcgaggacggagaggctgacgagcccgcgcgggagccaggtccagatctcggcgcggcg
ggggcggagagcgaagacgagggcgcgcagttgggagctgtccatggtgtcgcggagatccaggtccgggggcagggttctgag
gttgacctcgtagaagcgggtgagggcgtgcttgagatgcagatggtacttgatttctacgggtgagttggtggccgtgtccacgcatt
gcatgagcccgtagctgcgcggggccacgaccgtgccgcggtgcgcttttagaagcggtgtcgcggacgcgctcccggcggcag
cggcggttccggccccgcgggcaggggcggcagaggcacgtcggcgtggcgctcgggcaggtcccggtgctgcgccctgagag
cgctggcgtgcgcgacgacgcggcggttgacatcctggatctgccgcctctgcgtgaagaccacgggccccgtgactttgaacctga
aagacagttcaacagaatcaatctcggcgtcattgacggcggcctgacgcaggatctcttgcacgtcgcccgagttgtcctggtaggc
gatctcggacatgaactgctcgatctcctcctcctggagatcgccgcggcccgcgcgctccacggtggcggcgaggtcattcgagat
gcgacccattagctgcgagaaggcgcccaggccgctctcgttccagacgcggctgtagaccacgtccccgtcggcgtcgcgcgcg
cgcatgaccacctgcgcgaggttgagctccacgtgccgcgtgaagacggcgtagttgcgcaggcgctggaagaggttgttgagggt
ggtggcgatgtgctcggtgacgaagaagtacatgatccagcggcgcaggggcatctcgctgatgtcgccgatggcctccagcctttc
catggcctcgtagaaatccacggcgaagttgaaaaactgggcgttgcgggccgagaccgtgagctcgtcttccaggagccggatga
gctcggcgatggtggcgcgcacctcgcgctcgaaatccccggggacctcctcctcttcctcttcttccatgacgacctcttcttctatttct
tcctctgggggcggtggtggtggcggggcccgacgacgacggcgacgcaccgggagacggtcgacgaagcgctcgatcatctcc
ccgcggcggcgacgcatggtttcggtgacggcgcgaccccgttcgcgaggacgcagcgtgaagacgccgccggtcatctcccggt
aatggggcgggtccccgttgggcagcgatagggcgctgacgatgcatcttatcaattgcggtgtaggggacgtgagcgcgtcgagat
cgaccggatcggagaatctttcgaggaaagcgtctagccaatcgcagtcgcaaggtaagctcaaacacgtagcagccctgtggacg
ctgttagaattgcggttgctgatgatgtaattgaagtaggcgtttttaaggcggcggatggtggcgaggaggaccaggtccttgggtcc
cgcttgctggatgcggagccgctcggccatgccccaggcctggccctgacaccggctcaggttcttgtagtagtcatgcatgagccttt
caatgtcatcactggcggaggcggagtcttccatgcgggtgaccccgacgcccctgagcggctgcacgagcgccaggtcggcgac
gacgcgctcggcgaggatggcctgttgcacgcgggtgagggtgtcctggaagtcatccatgtcgacgaagcggtggtaggccccg
gtgttgatggtgtaggtgcagttggccatgagcgaccagttgacggtctgcaggccgggttgcacgacctcggagtacctgagccgc
gagaaggcgcgcgagtcgaagacgtagtcgttgcaggtgcgcacgaggtactggtagccgactaggaagtgcggcggcggctgg
cggtagagcggccagcgctgggtggccggcgcgcccggggccaggtcctcgagcatgaggcggtggtagccgtagaggtagcg
ggacatccaggtgatgccggcggcggtggtggaggcgcgcgggaactcgcggacgcggttccagatgttgcgcagcggcaggaa
ataatccatggtcggcacggtctggccggtgagacgcgcgcagtcattgacgctctagaggcaaaaacgaaagcggttgagcgggc
tcttcctccgtagcctggcggaacgcaaacgggttaggccgcgtgtgtaccccggttcgagtcccctcgaatcaggctggagccgcg
actaacgtggtattggcactcccgtctcgacccgagcccgatagccgccaggatacggcggagagccctttttgccgaccgagtggg
gtcgctagacttgaaagcggccgaaaaccccgccgggtagtggctcgcgcccgtagtctggagaagcatcgccagggttgagtcgc
ggcagaacccggttcgcggacggccgcggcgagcgggacttggtcaccccgccgatttaaagacccacagccagccgacttctcc
agttacgggagcgagcccccttttttctttttgccagatgcatcccgtcctgcgccaaatgcgtcccacccccccggcgaccaccgcga
ccgcggccgtaacaggcgccggcgctagccagccacagacagagatggacttggaagagggcgaagggctggcgagactggg
ggcgccgtccccggagcgacacccccgcgtgcagctgcagaaggacgtgcgcccggcgtacgtgcctgcgcagaacctgttcag
ggaccgcagcggggaggagcccgaggagatgcgcgactgccggtttcgggcgggcagggagctgcgcgagggcctggaccgc
cagcgcgtgctgcgcgacgaggatttcgagccgaacgagcagacggggatcagccccgcgcgcgcgcacgtggcggcggccaa
cctggtgacggcctacgagcagacggtgaagcaggagcgcaacttccaaaagagtttcaacaaccacgtgcgcacgctgatagcgc
gcgaggaggtggccctgggcctgatgcacctgtgggacctggcggaggccatcgtgcagaacccggacagcaagcctctgacgg
cgcagctgttcctggtggtgcagcacagcagggacaacgaggcgttcagggaggcgctgctgaacatcgccgagcccgagggtcg
ctggctgctggagctgatcaacatcttgcagagcatcgtagtgcaggagcgcagcctgagcctggccgagaaggtggcggcgatca
actactcggtgctgagcctgggcaagttttacgcgcgcaagatttacaagacgccgtacgtgcccatagacaaggaggtgaagatag
acagcttttacatgcgcatggcgctcaaggtgctgacgctgagcgacgacctgggcgtgtatcgcaacgaccgcatccacaaggccg
tgagcacgagccggcggcgcgagctgagcgaccgcgagctgatgctgagcctgcgccgggcgctggtagggggcgccgccgg
cggcgaggagtcctacttcgacatgggggcggacctgcattggcagccgagccggcgcgccttggaggccgcctacggtccagag
gacttggatgaggatgaggaagaggaggaggatgcacccgttgcggggtactgacgcctccgtgatgtgtttttagatgtcccagcaa
gccccggaccccgccataagggcggcgctgcaaagccagccgtccggtctagcatcggacgactgggaggccgcgatgcaacgc
atcatggccctgacgacccgcaaccccgagtcctttagacaacagccgcaggccaacagactctcggccattctggaggcggtggtc
ccctctcggaccaaccccacgcacgagaaggtgctggcgatcgtgaacgcgctggcggagaacaaggccatccgtcccgacgag
gccgggctggtgtacaacgccctgctggagcgcgtgggccgctacaacagcacgaacgtgcagtccaacctggaccggctggtga
cggacgtgcgcgaggccgtggcgcagcgcgagcggttcaagaacgagggcctgggctcgctggtggcgctgaacgccttcctgg
cgacgcagccggcgaacgtgccgcgagggcaggacgattacaccaactttatcagcgcgctgcggctgatggtgaccgaggttcc
ccagagcgaggtgtaccagtcgggcccggactactttttccagacaagccggcagggcctgcagacggtgaacctgagtcaggcttt
caagaacctgcgcgggctgtggggcgtgcaggcgcccgtgggcgaccggtcgacggtgagcagcttgctgacgcccaactcgcg
gctgctgctgctgctgatcgcgcccttcaccgacagcggcagcgtgaaccgcaactcgtacctgggccacctgctgacgctgtaccg
cgaggccataggccaggcgcaggtggacgagcagaccttccaggagatcacgagcgtgagccgcgcgctggggcagaacgaca
ccgacagtctgagggccaccctgaactttttgctgacaaatagacagcagaagatcccggcgcagtacgcactgtcggccgaggag
gaaaggatcctgagatatgtgcagcagagcgtagggctgttcctgatgcaggagggtgccacccccagcgccgcgctggacatgac
cgcgcgcaacatggaacctagcatgtacgccgccaaccggccgttcatcaataagctgatggactacctgcaccgcgcggcggcca
tgaacacggactactttacaaacgccatattgaacccgcactggcttccgccgccggggttctacacgggcgagtacgacatgcccg
accccaacgacgggttcctgtgggacgacgtggacagcgcggtgttctcgccgacctttcaaaagcgccaggaggcgccgccgag
cgagggcgcggtggggaggagcccctttcctagcttagggagtttgcatagcttgccgggctcggtgaacagcggcagggtgagcc
ggccgcgcttgctgggcgaggacgaatacctgaacgactcgctgctgcagccgccgcgggtcaagaacgccatggccaataacgg
gatagagagtctggtggacaaactgaaccgctggaagacctacgctcaggaccatagggagcctgcgcccgcgccgcggcgaca
gcgccacgaccggcagcggggcctggtgtgggacgacgaggactcggccgacgatagcagcgtgttggacttgggcgggagcg
gtggggccaacccgttcgcgcatctgcagcccagactggggcggcggatgttttgaatgcaaaataaaactcaccaaggccatagcg
tgcgttctcttccttgttagagatgaggcgtgcggtggtgtcttcctctcctcctccctcgtacgagagcgtgatggcgcaggcgaccct
ggaggttccgtttgtgcctccgcggtatatggctcctacggagggcagaaacagcattcgttactcggagctggctccgcagtacgac
accactcgcgtgtacttggtggacaacaagtcggcggacatcgcttccctgaactaccaaaacgaccacagcaacttcctgaccacg
gtggtgcagaacaacgatttcacccccgccgaggccagcacgcagacgataaattttgacgagcggtcgcggtggggcggtgatct
gaagaccattctgcacaccaacatgcccaatgtgaacgagtacatgttcaccagcaagtttaaggcgcgggtgatggtggctagaaag
catcccaaagatgtagatgccagtgatttaagcaaggatatcttagagtataagtggtttgagtttaccctgcccgagggcaacttttccg
agaccatgaccatagacctgatgaacaacgccatcttggaaaactacttgcaagtggggcggcagaatggcgtgctggagagcgata
tcggagtcaagtttgacagcaggaatttcagactgggctgggacccggtgaccaagctggtgatgccaggggtctacacctacgagg
ccttccacccggacgtggtgctactgccgggctgcggggtggacttcaccgagagccgcctgagcaacctcctgggcattcgcaag
aagcaaccttttcaagagggcttcagaatcatgtatgaggatctagaagggggtaacatccccgctctcctggataccaaaaaatatctg
gatagcaagaaggaacttgaggatgctgccaaggaagctgcaaagcaacagggagatggtgctgtcactagaggcgatacccacct
cactgtagctcaagaaaaagcagctgaaaaggagctagtgatcgtaccaattgaaaaggatgagagcaacagaagttacaacctgat
caaggacacccatgacaccctgtaccgaagctggtacctgtcctatacctacggggaccccgagaagggggtgcagtcgtggacgc
tgctcaccaccccggacgtcacctgcggcgcggagcaagtctactggtcgctgccggacctcatgcaagaccccgtcaccttccgct
ctacccagcaagtcagcaactaccccgtggtcggcgccgagctcatgcccttccgcgccaagagcttttacaacgacctcgccgtcta
ctcccagctcatccgcagctacacctccctcacccacgtcttcaaccgcttccccgacaaccagatcctctgccgcccgcccgcgccc
accatcaccaccgtcagtgaaaacgtgcctgctctcacagatcacgggacgcttccgctgcgcagcagtatccgcggagtccagcga
gtgaccgtcactgacgcccgtcgccgcacctgtccctacgtctacaaggccctgggcatagtcgcgccgcgcgtgctctccagtcgc
accttctaaaaaatgtctattctcatctcgcccagcaataacaccggctggggtcttactaggcccagcaccatgtacggaggagccaa
gaagcgctcccagcagcaccccgtccgcgtccgcggtcacttccgcgctccctggggagcttacaagcgggggcgcactgccacc
gccgccgccgtgcgcaccaccgtcgacgacgtcatcgactcggtggtcgccgacgcgcgcaactacacccccgccccctccaccg
tggacgcggtcatcgacagcgtggtggccgacgcgcgcgactatgccagacgcaagagccggcggcgacggatcgccaggcgc
caccggagcacgcccgccatgcgcgccgcccgggctctgctgcgccgcgccagacgcacgggccgccgggccatgatgcgagc
cgcgcgccgcgccgccactgcaccccccgcaggcaggactcgcagacgagcggccgccgccgctgccgcggccatctctagcat
gaccagacccaggcgcggaaacgtgtactgggtgcgcgactccgtcacgggcgtgcgcgtgcccgtgcgcacccgtcctcctcgt
ccctgatctaatgcttgtgtcctcccccgcaagcgacgatgtcaaagcgcaaaatcaaggaggagatgctccaggtcgtcgccccgg
agatttacggacccccggaccagaaaccccgcaaaatcaagcgggttaaaaaaaaggatgaggtggacgagggggcagtagagtt
tgtgcgcgagttcgctccgcggcggcgcgtaaattggaaggggcgcagggtgcagcgcgtgttgcggcccggcacggcggtggt
gttcacgcccggcgagcggtcctcggtcaggagcaagcgtagctatgacgaggtgtacggcgacgacgacatcctggaccaggcg
gcggagcgggcgggcgagttcgcctacgggaagcggtcgcgcgaagaggagctgatctcgctgccgctggacgaaagcaaccc
cacgccgagcctgaagcccgtgaccctgcagcaggtgctgccccaggcggtgctgctgccgagccgcggggtcaagcgcgagg
gcgagagcatgtacccgaccatgcagatcatggtgcccaagcgccggcgcgtggaggacgtgctggacaccgtgaaaatggatgt
ggagcccgaggtcaaggtgcgccccatcaagcaggtggcgccgggcctgggcgtgcagaccgtggacattcagatccccaccga
catggatgtcgacaaaaaaccctcgaccagcatcgaggtgcagaccgacccctggctcccagcctccaccgctaccgtctccactttt
accgccgccacggctaccgagcctcccaggaggcgaagatggggcgccgccagccggctgatgcccaactacgtgttgcatcctt
ccatcatcccgacgccgggctaccgcggcacccggtactacgccagccgcaggcgcccagccgccaaacgccgccgccgcayt
gccacccgccgccgtmtggcccccgcccgcgtgcgccgcgtaaccacgcgccggggccgctcgytcgttctgcccaccgtgcgc
taccaccccagcatcctttaatccgtgtgctgtgatactgttgcagagagatggctctcacttgccgcctgcgcatccccgtcccgaatta
ccgaggaagatcccgccgcaggagaggcatggcaggcagcggcctgaaccgccgccggcggcgggccatgcgcaggcgcctg
agtggcgggtttctgcccgcgctcatccccataatcgccgcggccatcggcacgatcccgggcatagcttccgttgcgctgcaggcgt
cgcagcgccgttgatgtgcgaataaagcctctttagactctgacacacctggtcctgtatatttttagaatggaagacatcaattttgcgtc
cttggctccgcggcacggcacgcggccgttcatgggcacctggaacgagatcggcaccagccagctgaacgggggcgccttcaat
tggagcagtgtctggagcgggcttaaaaatttcggctcgacgctccggacctatgggaacaaggcctggaatagtagcacggggca
gttgttaagggaaaagctcaaagaccagaacttccagcagaaggtggtggacgggctggcctcgggcattaacggggtggtggaca
tcgcgaaccaggccgtgcagcgcgagataaacagccgcctggacccgcggccgcccacggtggtggagatggaagatgcaactc
ttccgccgcccaaaggcgagaagcggccgcggcccgacgcggaggagacgatcctgcaggtggacgagccgccctcgtacgag
gaggccgtcaaggccggcatgcccaccacgcgcatcatcgcgccgctggccacgggtgtaatgaaacccgccacccttgacctgc
ctccaccacccacgcccgctccaccgaaggcagctccggttgtgcaggcccctccggtggcgaccgccgtgcgccgcgtccccgc
ccgccgccaggcccagaactggcagagcacgctgcacagtatcgtgggcctgggagtgaaaagtctgaagcgccgccgatgctatt
gagagaggaaagaggacactaaagggagagcttaacttgtatgtgccttaccgccagagaacgcgcgaagatggccaccccctcg
atgatgccgcagtgggcgtacatgcacatcgccgggcaggacgcctcggagtacctgagcccgggtctggtgcagtttgcccgcgc
caccgacacgtacttcagcctgggcaacaagtttaggaaccccacggtggccccgacccatgatgtgaccacggaccggtcccagc
gtctgacgctgcgcttcgtgcccgtggatcgcgaggacaccacgtactcgtacaaggcgcgcttcactctggccgtgggcgacaacc
gggtgctagacatggccagcacgtactttgacatccgcggcgtcctggaccgcggtcccagcttcaaaccctactcgggcacggctt
acaacagtttggcccccaagggcgcccccaactccagtcagtggactgacaaagaacggcaaaatggtggacaaccacccactaca
aaagatgttacaaaaacattcggagtagcagccaggggagggcttcatattactgataaaggactacaaataggagaagatgaaaata
acgaggatggtgaagaagagatatatgcagacaaaactttccagccagaacctcaagtaggagaggaaaactggcaagatactgat
gttttctatggcggcagagcgcttaaaaaggaaaccaaaatgaaaccatgctatggctcttttgccagacctaccaatgaaaaaggagg
tcaagctaaatttttaaatggcgaaaacggtcaaccttctaaagatcaagatattacattagctttctttgatcttaaacaaaatgacactgg
aactactcaaaaccagccagatgttgtcatgtacactgaaaatgtgtatttggaaaccccagacacccatgtggtgtacaaacctggca
aggaagatacaagctccgctgctaaccttacacaacagtccatgcccaacaggcccaactacattggtttcagggacaactttgtggg
gctcatgtattacaacagcactggcaacatgggtgtgctggctggtcaggcctctcagttgaatgctgtggttgacttgcaagacagaaa
caccgagctgtcttatcagctcttgctagattctctgggtgacagaaccagatactttagcatgtggaattctgcggtggacagctatgat
ccagatgtcaggatcattgagaatcacggtgttgaagatgagcttccaaattattgcttcccactggatggatctggcagcaataccgca
tatcaaggtgttaaatatgaaaacggagctggcaatggaagctggaaagtagatggcgaagttgcttctcagaatcagatcgccaagg
gtaatctgtatgccatggagataaaccttcaggccaacctgtggaagagttttctgtactcgaacgtggcgctgtatctaccagactccta
caagtacacgccggccaacatcacgctgcccaccaacaccaacacctacgagtacatgaacggccgcgtggtggcaccctcgctg
gtggatgcctatgtcaacatcggtgcccgctggtcgctggaccccatggacaacgtcaaccccttcaaccaccaccgcaacgcgggt
ctgcgctaccgctccatgcttctgggcaacggccgctacgtgcccttccacatccaagtgccccaaaagttctttgccatcaagaacct
gctcctgcttcccggttcctacacctacgagtggaacttccgcaaggatgtcaacatgatcctgcaaagttccctcggcaacgacctgc
gcgtcgacggcgcctccgtccgcttcgacagcgtcaacctctatgccaccttcttccccatggcgcacaacaccgcctccacccttga
agccatgctgcgcaacgacaccaacgaccagtccttcaacgactacctctcggccgccaacatgctctacccaatcccggccaaggc
caccaacgtgcccatctccatcccctcgcgcaactgggccgccttccgcggctggagtttcacccggctcaagaccaaggaaactcc
ctccctcggctcgggtttcgacccctactttgtctactcgggctccattccctacctcgacggaaccttctacctcaaccacaccttcaag
aaggtctccatcatgttcgactcctcggtcagctggcccggcaacgaccggctgctcacgccgaacgagttcgagatcaagcgcagc
gtcgacggggagggctacaacgtggcccaatgcaacatgactaaggactggttcctcgtccagatgctctctcattacaacattggcta
ccagggcttctacgtgcctgacggttacaaggaccgcatgtactccttcttccgcaacttccagcccatgagcaggcaggtggtcgatg
agatcaactacaaggactacaaggccgtcaccctgcccttccagcacaacaactcgggcttcaccggctacctcgcacccaccatgc
gtcaggggcagccataccccgccaacttcccctacccgctcatcggccagacagccgtgccctccgtcacccagaaaaagttcctct
gcgacagggtcatgtggcgcatccccttctccagcaacttcatgtccatgggcgccctcaccgacctgggtcagaacatgctctacgc
caactcggcccacgcgctcgacatgaccttcgaggtggaccccatggatgagcccaccctcctctatcttctcttcgaagttttcgacgt
ggtcagagtgcaccagccgcaccgcggcgtcatcgaggccgtctacctgcgcacgcccttctccgccggaaacgccaccacataa
gcatgagcggctccagcgaacgagagctcgcggccatcgtgcgcgacctgggctgcgggccctactttttgggaacccacgacaa
gcgcttccctggcttcctcgccggcgacaagctggcctgcgccatcgtcaacacggccggccgcgagaccggaggcgtgcactgg
ctcgccttcggctggaacccgcgctcgcgcacctgctacatgttcgacccctttgggttctcggaccgccggctcaagcagatttacag
cttcgagtacgaggccatgctgcgccgcagcgccctggcctcctcgcccgaccgctgtctcagcctcgagcagtccacccagaccgt
gcaggggcccgactccgccgcctgcggacttttctgttgcatgttcttgcatgccttcgtgcactggcccgaccgacccatggacggg
aaccccaccatgaacttgctgacgggggtgcccaacggcatgctacaatcgccacaggtgctgcccaccctccggcgcaaccagga
ggagctctaccgcttcctcgcgcgccactccccttactttcgatcccaccgcgccgccatcgaacacgccaccgcttttgacaaaatga
aacaactgcgtgtatctcaataaacagcacttttattttacatgcactggagtatatgcaagttatttaaaagtcgaaggggttctcgcgctc
gtcgttgtgcgccgcgctggggagggccacgttgcggtactggtacttgggaagccacttgaactcggggatcaccagtttgggaac
cggaatctcggggaaggtctcgctccacatgcgccggctcatctgcagggcgcccagcatgtccggggcggagatcttgaaatcgc
agttgggaccggtgctctgcgcgcgcgagttgcggtacacggggttgcagcactggaacaccatcagactggggtacttcacactgg
ccagcacgctcttgtcggtgatctgatccttgtcaaggtcctcggcgttgctcaggccaaacggggtcatcttgcacagctggcggccc
aggaagggcacgctctgaggcttgtggttacactcgcagtgaatgggcattagcatcatccccgcgccgcgctgcatattcgggtaga
gggccttgacaaaggccgagatctgtttgaaagcttgctgggccttggctccctcgctgaaaaacagcccgcagctcttcccgctgaa
ctggttattcccgcaaccggcatcctgcacgcagcagcgcgcgtcatggctggtcagttgcaccacgctccgtccccagcggttctgg
gtcaccttggccttgctgggttgctccttcagcgcgcgctgtccgttctcgctggtcacatccatctccaccacgtggtctttgtggatcat
caccgttccatgcagacacttgagctggccttccacctcggtgcagccgtgatcccacagggcgcatccggtgcactcccagttcttat
gcgcgatcccgctgtggctgaagatgtaaccttgcaacaggcgacccatgacggtgctaaaggctttctgggtggtgaaggtcagttg
cagaccgcgggcctcctcgttcatccaggtctggcacatcttctggaagatctcggtctgctctggcatgagcttgtaagcatcgcgca
ggccgctgtcgacgcggtagcgttccatcagcacgttcatggtatccatgcccttctcccaggacgagaccagaggcagactcaggg
ggttgcgcacgttcaggacaccgggggtcgcgggctcgacgatgcgttttccgtccttgccttccttcaacagaaccggaggctggct
gaatcccactcccacgatcacggcgtcttcctggggcatctcttcgtctgggtctacctttgtcacatgcttggtctttctggcttgcttctttt
ttggagggctgtccacggggaccacgtcctcctccgaagacccggagcccacccgctgatactttcggcgcttggtgggcagagga
ggtggcggcgaggggctcctctcctgctccggcggatagcgcgccgacccgtggccccggggcggartggcctctcgctccatga
accggcgcacgtcctgactgccgccggccattgtttcctaggggaagatggaggagcagccgcgtaagcaggagcaggaggagg
acttaaccacccacgagcaacccaaaatcgagcaggacctgggcttcgaagagccggctcgtctagaacccccacaggatgaaca
ggagcacgagcaagacgcaggccaggaggagaccgacgctgggctcgagcatggctacctgggaggagaggaggatgtgctgc
tgaaacacctgcagcgccagtccctcatcctccgggacgccctggccgaccggagcgaaacccccctcagcgtcgaggagctgtgt
cgggcctacgagctcaacntcttctcgccgcgcgtgccccccaaacgccagcccaacggcacctgcgagcccaacccgcgtctca
acttctatcccgtctttgcggtccccgaggcccttgccacctatcacatctttttcaagaaccaaaagatccccgtctcctgccgcgccaa
ccgcacccgcgccgacgcgctcctcgctctggggcccggcgcgcgcatacctgatatcgcttccctggaagaggtgcccaagatctt
cgaagggctcggtcgggacgagacgcgcgcggcgaacgctctgaaagaaacagcagaggaagagggtcacactagcgccctgg
tagagttggaaggcgacaacgccaggctggtcgtgctcaagcgcagcgtcgagctcacccacttcgcctaccccgccgttaacctcc
cgcccaaggtcatgcgtcgcatcatggatcagcttatcatgccccacatcgaggccatcgatgagacccaagagcagcgccccgag
gacgcccggcccgtggtcagcgacgagatgctcgcgcgctggctcgggacccgcgacccccaggctttggaacagcggcgcaag
ctgatgctggccgtagtcctggtcaccctcgagctcgaatgcatgcgccgcttcttctgcgaccccgagaccctgcgcaaggtcgagg
agaccctgcactacactttcagacacggtttcgtcaggcaagcctgcaagatctccaacgtggagctgaccaacctggtctcctgcctg
gggatcctgcatgagaaccgcctggggcagacagtgctccactctaccctgaagggcgaggcacggcgggactatgtccgcgact
gcgtctttctctttctatgccacacatggcaagcagccatgggcgtgtggcagcagtgtctcgaggacgagaacctgaaggagctgga
caagcttcttgctagaaaccttaaaaagttgtggacgggcttcgacgagcgcaccgtcgcctcggacctggccgagatcgttttccccg
agcgcctgaggcatacgctgaaaggcgggctgcccgacttcatgagccagagcatgttgcaaaactaccgcactttcattctcgagcg
ctcgggtatcctgcccgccacctgcaacgccttcccctccgactttgtcccgctgagctaccgcgagtgtcccccgccgctgtggagc
cactgctacctcttgcagctggctaactacatctcctaccactcggacgtgatcgaggacgtgagcggcgaggggctgctcgagtgcc
actgccgctgcaacctgtgctccccgcaccgctccctggtctgcaacccccagctcctgagcgagacccaggtcatcggtaccttcga
gctgcaaggtccggagaagtccaccgctccgctgaaactcacgccggggttgtggacttccgcgtacctgcgcaaatttgtacccga
agactaccacgcccatgagataaagttcttcgaggaccaatcgcgtccgcagcacgcggatctcacggcctgcgtcatcacccaggg
cgcgatcctcgcccaattgcatgccatccaaaaatcccgccaagagtttcttctgaaaaagggtagaggggtctacctggacccccag
acgggcgaggtgctcaacccgggtctcccccagcatgccgaggaagaagcaggagccgctagtggaggagatggaagaagaatg
ggacagccaggcagaggaggacgaatgggaggaggagacagaggaggaagaattggaagaggtggaagaggagcaggcaac
agagcagcccgtcgccgcaccatccgcgccggcagccccggcggtcacggatacaacctccgctccggtcaagcctcctcgtaga
tgggatcgagtgaagggtgacggtaagcacaagcggcagggctaccgatcatggagggcccacaaagccgcgatcatcgcctgct
tgcaagactgcggggggaacatcgctttcgcccgccgctacctgctcttccaccgcggggtgaacatcccccgcaacgtgttgcatta
ctaccgtcaccttcacagctaagaaaaagcaagtaagaggagtcgccggaggaggcctgaggatcgcggcgaacgagccctcgac
caccagggagctgaggaaccggatcttccccactctttatgccatttttcagcagagtcgaggtcagcagcaagagctcaaagtaaaa
aatcggtctctgcgctcgctcacccgcagttgcttgtaccacaaaaacgaagatcagctgcagcgcactctcgaagacgccgaggctc
tgttccacaagtactgcgcgctcactcttaaagactaaggcgcgcccacccggaaaaaaggcgggaattacctcatcgccaccatga
gcaaggagattcccaccccttacatgtggagctatcagccccagatgggcctggccgcgggcgcctcccaggactactccacccgc
atgaactggctcagtgccggcccctcgatgatctcacgggtcaacggggtccgtaaccatcgaaaccagatattgttggagcaggcg
gcggtcacatccacgcccagggcaaagctcaacccgcgtaattggccctccaccctggtgtatcaggaaatccccgggccgactac
cgtactacttccgcgtgacgcactggccgaagtccgcatgactaactcaggtgtccagctggccggcggcgcttcccggtgcccgct
ccgcccacaatcgggtataaaaaccctgatgatccgaggcagaggcacacagctcaacgacgagttggtgagctcttcgatcggtct
gcgaccggacggagtgttccaactagccggagccgggagatcatccttcactcccaaccaggcctacctgaccttgcagagcagctc
ttcggagcctcgctccggaggcatcggaaccctccagttcgtggaggagtttgtgccctcggtctacttcaaccccttctcgggatcgc
caggcctctacccggacgagttcataccgaacttcgacgcagtgagagaagcggtggacggctacgactgaatgtcccatggtgact
cggctgagctcgctcggttgaggcatctggaccactgccgccgcctgcgctgcttcgcccgggagagctgcggactcatctactttga
gctgcccgaggagcaccccaacggccctgcacacggagtacggatcaccgtagagggcaccgccgagtctcacctggtcaggttc
ttcacccagcaacccttcctggtcgagcgggaccggggcgccaccacctacaccgtctactgcatctgtcctaccccaaagttgcatg
agaatttttgctgtactattgtggtgagtttaataaaagctgaactaagaacctactttggaatcccttgtcgtcatcaaatccacaagacc
atcaacttcacctttgaggaacaggtgaactttacctgcaagccacacaagaagtacgtcacctggttttaccagaacactactctagca
gtagccaacacctgctcgaacgacggtgttcttcttccaaacaatctcaccagtggactaactttctcagtgaaaagggcaaagctaatt
cttcatcgccctattgtagaaggaacttaccagtgtcagagcggaccttgcttccacagtttcactttggtgaacgttaccggcagcagca
cagtcgctccagaaactaaccttctttctgatactaacactcctaaaaccggaggtgagctctgggttccctctctgacagaggggggta
gtcatattgaagcggtcgggtatttgattttaggggtggtcctgggtgggtgcatagcggtgctatattaccttccttgctgggtcgaaatc
agggtatttatctgctgggtcagacattgtggggaggaaccatgaaggggctcttgctgattatcctttccctggttgggggtttactggc
ctgccacgaacagccacgatgtaacatcaccacaggcaatgagaggaacgactgctctgtagtgatcaaatgcgagcaccagtgtcc
tctcaacattacattcaagaataagaccatgggaaatgtatgggtgggattctggcaaccaggagatgagcagaactacacggtcacta
tccatggtagcgatggaaatcacactttcggtttcaaattcatttttgaagtcatgtgtgatatcacactgcatgtggctagacttcatggctt
gtggccccctaccaaggagaacatggttgggttttctttggcttttgtgatcatggcctgtgcaatgtcaggtctgctggtaggggctcta
gtgtggttcctgaagcgcaagcccaggtacggaaatgaggagaaggaaaaattgctataaatctttttctcttcgcagaaccatgaatac
tttgaccagtgtcgtgctgctctctcttttagttattaatgtggaatgtgccgatcctattctagttagtgtagattggggaaaaaatcttacatt
agagggtcctaaagaaacaccagttgaatggtggggtggaagaaacatacaacaactgtgcatagggaatcaaaccaaacataaag
agctaagtcacagatgtaatgtccagaacataactttactgatgtaaatactagttttaatggagactactttgggtttaaaaatgataacag
cggtatgaaacattataaagtcacagttataccccctaaaccctccactcggaaacctctttctcctccacactatgtaaacgcaactatg
gggcaaaacctaacattagtggggcctgcaaacattccagttacttggcttagtgaatatggcacgttgtgtgagggcaaaaaaattttg
cacaaagnaattaaatcacacctgtaacgaacagaacctcacgttactgtttgttaatatgacacacaacggaccatattttggctttgac
aaatacaacattgatagagagcagtatgaggtttctattattagtttgtttaaagttggcgctggacagaagaaaattgggaaaggacaga
aaaaggaggaaaagacaaaaccaaactctagtgatttgggacaaagacaatccagaccaaagaaaaaagatattgttgaagaggtcc
aaatcaaaacaggagaaaatcgaacccttgttggtccacctggaaaagttgattggattaaactttccagtggaaacaataatgttcttaa
gttgtgtaatggcgacaagtatattaaacacacatgtgatggtcaaaatttaacattaattaatgtgactagaatttatgacggaacttattat
ggttctagcaatgatggctcaagtcattacaaagttaccatctatgaattacacaaagttaataaaactaaatctatgcttaagccatacact
acaaaaagaactacagtgaatgcaacagatgacagtgctcacaaaattgctttgcagcaggaaaataatgggcaaacagaaaatgatc
aagaatcaaaaattccatctgctactgtggcaatcgtggtgggagtgattgcgggcttcataactataatcattgtcattctgtgctacatct
gctgccgcaagcgtcccagggcatacaataatatggtagacccactactcagatctatactgagactcagtcactttcatttcagaacc
atgaaggctttcacagcttgcgttctgattagcataattacacttagtttagcagcacctaaaccagaagtatatacacaagttaatgtcact
aggggtgggaatgctacactagatggaccatttaacaataacacatggacaagatatcatgatgatgggagaaaaaacggatggatg
aatatttgtaaatggtcagacccatcatacacatgtcatagtaatggaagccttagtatttttgctttcaacattagttcaggtaaatataaagt
tcaaagttacactaacagttataatggattagatggttatgaaaaacttgaagttaaaatgtttaatctaacagtaattgagcctccaaccac
tagagcacccaccacagttaggacaactaaggaaacaacacagcctaccactgtacccactacacatccaaccaccacagtcagtac
aactattgagaccactactcatactacacagctagacacaacagtgcagaatactactttactgattgaatttttactaagagggaatgaa
agtactactgatcagacagaggctacctcaagtgccttcagcagtactgcaaatttaacttcgcttgcttggactaatgaaaccggagtat
cattgatgcatggccagccttactcaggtttggatattcaaattacttttctggttgtctgtgggatctttattcttgtggttcttctgtactttgtc
tgctgcaaagccagagagaaatctagtaggcccatctacaggccagtaatcggggaacctcagcctctccaagtggaagggggtcta
aggaatcttctcttctctttttcagtatggtgatcagccatgattcctaggttcttcctatttaacatcctcttctgtctcttcaacatctgcgctg
ccttcgcggccgtctcgcacgcctcgcccgactgtctcgggcccttccccacctacctcctctttgccctgctcacctgcacctgcgtct
gcagcattgtctgcctggtcgtcaccttcctgcagctcatcgactggtgctgcgcgcgttacaattatctccaccacagtcccgaataca
gggacaagaacgtagccagaatcttaaggctcatctgaccatgcagactctgctgatactgctatccctcctctcccctgcccttgctga
ctgtaaatttgcggacatatggaatttcttagactgttatcaagagaaaatggatatgccttcctattacttggtgattgtgggtgtagtcatg
gtctgctcctgcactttctttgctatcatgatctacccctgttttgatctcggctggaactctgttgaggcattcacatacacactagaaagca
gttcactagcctccacgccgccacccacaccgcctccccgcagaaatcagttccccctgattcagtacttagaagagccccctccccg
gcccccttccactgttagctactttcacataaccggcggcgatgactgaccaccacctggacctcgagatggacggccaggcctccga
gcagcgcatcctgcaactgcgcgtccgtcagcagcaggagcgggccgccaaggagctcctcgatgccatcaacatccaccagtgc
aagaagggcatcttctgcctggtcaaacaggcaaagatcacctacgagctcgtgtccaacggcaaacagcatcgcctcacctatgag
atgccccagcagaagcagaagttcacctgcatggtgggcgtcaaccccatagtcatcacccagcagtcgggcgagaccagcggctg
catccactgctcctgcgaaagccccgagtgcatctactccctcctcaagaccctttgcggacttcgcgacctcctccccatgaactgatt
gattaaagcccagaaaccaatcaaacccccttccccatcaccccaaataacaatcattggaaataatcattcaataaagatcacttacttg
aaatctgaaagtatgtctctggtgtagttgttcagcagcacctcggtaccctcctcccagctctggtactccagtccccggcgggcggc
gaacttcctccacaccttgaaagggatgtcaaattcctggtccacaattttcattgtatccctctcagatgtcaaagaggctccgggtgga
agatgacttcaaccccgtctacccctatggctacgcgcggaatcagaatatcccatcctcactcccccctttgtctcctccgatggattc
caaaacttcccccctggggtcctgtcactcaaactggctgatccaatcgccatcgccaatgggaatgtctcactcaaggtgggagggg
gactcactgtagaacaacagtctggaaaactgagtgtggatactaaggcacccttgcaagttgcaaatgacaacaaattggagctatctt
atgatgatccatttaaggtagagaataacaaacttggaattaaagctggccatggtttagcagttgtaactaaagaaaacacaagtcttcc
tagtctagttggaacacttgtagttttaactggaaaaggaataggtactggatcaagtgcacatggaggaactattgatgtaagacttggt
gaaggaggtgggttatcatttgatgaaaaaggagacttagtagcttgggacaaaaaaaatgatacacgcaccctttggacaacacctgt
ccttctccaaattgcaaagttgaaacagcaagagactcaaagctaaccttagcacttacaaaatgtggtagtcaaattttggccactgtat
ctttacttgttgttacgggcaaatatgctattataagtgacacagtcaacccaaagcagttctctattaagttactgtttaatgacaagggtgt
tttgttaagkgactcaaatcttgatgggacatattggaactatagaagcaacaataacaacataggcactccttataaagaggctgttggt
tttatgccaagcacaacagcttatcctaagccaaccaacaacaccagcacagatccggataaaaaagtgagtcaaggtaaaaataaaa
ttgtaagcaatatttatcttggaggagaggtatatcaaccaggatttattgttgttaaatttaatcaggaaactgatgccaattgtgcatactct
attacatttgattttggatggggtaaggtgtataaggatcctataccatatgatacctcttcttttactttctcatatatcgctcaagaatgaaag
accaataaacgtgtttttcattgaaaattttcatgtatctttattgatttttacaccagcacgggtagtcagtctcccaccaccagcccatttca
cagtgtaaacaattctctcagcacgggtggccttaaataggggaatgttctgattagtgcgggaactggatttagtgtctataatccacac
agtttcctggcgagccaaacggggatcggtgattgagatgaagccgtcctctgaaaagtatccaagcgggcctcacagtccaaggtc
acagtctggtggaatgagaagaacgcacagattcatactcggaaaacaggatgggtctgtgcctctccatcagcgccctcaacagtct
ctgccgccggggctcggtgcggctgctgcagatgggatcgggatcgcaagtctctctgactatgatccccacagccttcagcatcagt
ctcctggtgcgtcgggcacagcacctcatcctgatctcgctcatgttctcacagtaagtgcagcacataatcaccatgttattcagcagcc
cataattcagggtgctccagccaaagctcatgttggggatgatggaacccacgtgaccatcataccaaatgcggcagtatatcaggtg
cctgcccctcatgaacacactgcccatatacatgatctctagggcatgtttctgttcacaatctggcggtaccaggggaagcgctggttg
aacatgcacccgtaaatgactctcctgaaccacacggccagcarggtgcctcccgcccggcactgcagggagcccggggacgaac
agtggcaatgcaggatccagcgntcgnacccgctcaccatctgagctctcaccaagtccagggtagcggggcacaggcacactga
catacatctttttaaaatttttatttcctctggggtcaggatcatatcccaggggactggaaactcttggagcagggtaaagccagcagca
catggtaatccacggacagaacttacattatgatattcagcatgatcacaatcgggcagcagggggtgttgttcagtcagtgaggccct
ggtctcctcctcagatcgtggtaaacgggccctgcggtatggatgatggcggagcgaggtcgattgttcctcggtgctcattgtagtgc
accctcttgcgtaccttgtcgtacttctgccagcagaaagtggcccgggaacagcagatacccctcctccgtccgtcctttcgctgctgc
cgctcagtcatccaactgaagtacatccattcccgaaggttctggagaagttcctctgcatctgatgaaacaaaaagcccgtccatgcg
aattcccctcatcacatcagccaggactctgtaggccatccccatccagttaatgctgccttgtctatcattcagagggggcggtggcag
gattggaagaaccattatttttttactccaaacggtcgcgcagcaatataaaattcaggtcacggaggtggcacctctctcctccactgttt
tggtggaaacagacagccaaatcaaaaattatgcgattctcaaggtgctctactgtggcttccagcagaggctccacacgtacatccag
aaacaacagcacattaaaagcgggcccgccatcctgctcmtcaatcatcatattacagtcctgaaccatccccaggtaattttcgtttttc
cagccttgaattatcgstacaa-3′

### Specific Example 5

The nucleic acid encoding the Ad-36 hexon protein (SEQ ID NO.: 30) is as follows:

5′atggccaccc cctcgatgat gccgcagtgg gcgtacatgc
acatcgccgg gcaggacgcc tcggagtacc tgagcccggg
tctggtgcag tttgcccgcg ccaccgacac gtacttcagc
ctgggcaaca agtttaggaa ccccacggtg gccccgaccc
atgatgtgac cacggaccgg tcccagcgtc tgacgctgcg
cttcgtgccc gtggatcgcg aggacaccac gtactcgtac
aaggcgcgct tcactctggc cgtgggcgac aaccgggtgc
tagacatggc cagcacgtac tttgacatcc gcggcgtcct
ggaccgcggt cccagcttca aaccctactc gggcacggct
tacaacagtt tggcccccaa gggcgccccc aactccagtc
agtggactga caaagaacgg caaaatggtg gacaaccacc
cactacaaaa gatgttacaa aaacattcgg agtagcagcc
aggggagggc ttcatattac tgataaagga ctacaaatag
gagaagatga aaataacgag gatggtgaag aagagatata
tgcagacaaa actttccagc cagaacctca agtaggagag
gaaaactggc aagatactga tgttttctat ggcggcagag
cgcttaaaaa ggaaaccaaa atgaaaccat gctatggctc
ttttgccaga cctaccaatg aaaaaggagg tcaagctaaa
tttttaaatg gcgaaaacgg tcaaccttct aaagatcaag
atattacatt agctttcttt gatcttaaac aaaatgacac
tggaactact caaaaccagc cagatgttgt catgtacact
gaaaatgtgt atctggaaac cccagacacc catgtggtgt
acaaacctgg caaggaagat acaagctccg ctgctaacct
tacacaacag tccatgccca acaggcccaa ctacattggt
ttcagggaca actttgtggg gctcatgtat tacaacagca
ctggcaacat gggtgtgctg gctggtcagg cctctcagtt
gaatgctgtg gttgacttgc aagacagaaa caccgagctg
tcatatcagc tcttgctaga ttctctgggt gacagaacca
gatactttag catgtggaat tctacggtgg acagctatga
tccagatgtc aggatcattg agaatcacgg tgttgaagat
gagcttccaa attattgctt cccactggat ggatctggca
gcaataccgc atatcaaggt gttaaatatg aaaacggagc
tggcaatgga agctggaaag tagatggcga agttgcttct
cagaatcaga tcgccaaggg taatctgtat gccatggaga
taaaccttca ggccaacctg tggaagagtt ttctgtactc
gaacgtggcg ctgtatctac cagactccta caagtacacg
ccggccaaca tcacgctgcc caccaacacc aacacctacg
agtacatgaa cggccgcgtg gtggcaccct cgctggtgga
tgcctatgtc aacatcggtg cccgctggtc gctggacccc
atggacaacg tcaacccctt caaccaccac cgcaacgcgg
gtctgcgcta ccgctccatg cttctgggca acggccgcta
cgtgcccttc cacatccaag tgccccaaaa gttctttgcc
atcaagaacc tgctcctgct tcccggttcc tacacctacg
agtggaactt ccgcaaggat gtcaacatga tcctgcaaag
ttccctcggc aacgacctgc gcgtcgacgg cgcctccgtc
cgcttcgaca gcgtcaacct ctatgccacc ttattcccca
tggcgcgcaa caccgcctcc acccttgaag ccatgctgcg
caacgacacc aacgaccagt ccttcaacga ctacctctcg
gccgccaaca tgctctaccc aatcccggcc aaggccacca
acgtgcccat ctccatcccc tcgcgcaact gggccgcctt
ccgcggctgg agtttcaccc ggctcaagac caaggaaact
ccctccctcg gctcgggttt cgacccctac tttgtctact
cgggctccat tccctacctc gacggaacct tctacctcaa
ccacaccttc aagaaggtct ccatcatgtt cgactcctcg
gtcagctggc ccggcaacga ccggctgctc acgccgaacg
agttcgagat caagcgcagc gtcgacgggg agggctacaa
cgtggcccaa tgcaacatga ctaaggactg gttcctcgtc
cagatgctct ctcattacaa cattggctac cagggcttct
acgtgcctga gggttacaag gaccgcatgt actccttctt
ccgcaacttc cagcccatga gcaggcaggt ggtcgatgag
atcaactaca aggactacaa ggccgtcacc ctgcccttcc
agcacaacaa ctcgggcttc accggctacc tcgcacccac
catgcgtcag gggcagccat accccgccaa cttcccctac
ccgctcatcg gccagacagc cgtgccctcc gtcacccaga
aaaagttcct ctgcgacagg gtcatgtggc gcatcccctt
ctccagcaac ttcatgtcca tgggcgccct caccgacctg
ggtcagaaca tgctctacgc caactcggcc cacgcgctcg
acatgacctt cgaggtggac cccatggatg agcccaccct
cctctatctt ctcttcgaag ttttcgacgt ggtcagagtg
aacgccacca cataa-3′

### Specific Example 6

The nucleic acid encoding the Ad-36 fiber coat protein (SEQ ID NO.: 31) is as follows:

5′atgtcaaagaggctccgggtggaagatgacttcaaccccgtctacccc
tatggctacgcgcggaatcagaatatccccttcctcactcccccctttgt
ctcctccgatggattccaaaacttcccccctggggtcctgtcactcaaac
tggctgatccatgtctcactcaaggtgggagggggactcactgtagaaca
acagtctggaaaactgagtgtggatactaaggcacccttgcaagttgcaa
atgacaacaaattggagctatcttatgatgatccatttaaggtagagaat
aacaaacttggaattaaagctggccatggtttagcagttgtaactaaaga
aaacacaagtcttcctagtctagttggaacacttgtagttttaactggaa
aaggaataggtactggatcaagtgcacatggaggaactattgatgtaaga
cttggtgaaggaggtgggtatcatttgatgaaaaaggagacttagtagct
tgggacaaaaaaaatgatacacg
caccctttggacaacacctgatccttctccaaattgcaaagttgaaacag
caagagactcaaagctaaccttagcacttacaaaatgtggtagtcaaatt
ttggccactgtatctttacttgttgttacgggcaaatatgctattataag
tgacacagtcaacccaaagcagttctctattaagttactgtttaatgaca
agggtgattgttaagtgactcaaatcttgatgggacatattggaactata
gaagcaacaataacaacataggcactccttataaagaggctgttggtttt
atgccaagcacaacagcttatcctaagccaaccaacaacaccagcacaga
tccggataaaaaagtgagtcaaggtaaaaataaaattgtaagcaatatat
cttggaggagaggtatatcaaccaggatttattgttgttaaatttaatca
ggaaactgatgccaattgtgcatactctattacatttgatttggatgggg
taaggtgtataaggatcctataccatatgatacctcttctactttctcat
atatcgctcaagaatga-3′

The examples given above are merely illustrative and are not meant to be an exhaustive list of all possible embodiments, applications or modifications of the invention. Thus, various modifications and variations of the described methods and systems of the invention will be apparent to those skilled in the art without departing from the scope and spirit of the invention. Although the invention has been described in connection with specific embodiments, it should be understood that the invention as claimed should not be unduly limited to such specific embodiments. Indeed, various modifications of the described modes for carrying out the invention which are obvious to those skilled in cellular and molecular biology, chemistry, or in the relevant fields are intended to be within the scope of the appended claims.

The disclosures of all references and publications cited above are expressly incorporated by reference in their entirety to the same extent as if each were incorporated by reference individually.

1. A method for evaluating the safety and suitability of animals or meat for consumption by a consumer, said method comprising the steps of:
screening a sample from an animal designated for consumption to determine whether the animal is infected with a lipogenic adenovirus; and
taking corrective action if the animals is positive for lipogenic adenovirus infection.

2. The method of claim 1, further comprising taking corrective action if the animal is not infected with lipogenic adenovirus.

3. The method of claim 2, wherein the corrective action comprises administering a lipogenic adenovirus vaccine to the animal prior to processing its meat for consumption.

4. The method of claim 1, wherein the sample in said screening step is taken from the animal ante-mortem.

5. The method of claim 1, wherein the sample in said screening step is taken from the animal post-mortem.

6. The method of claim 1, wherein said corrective action in said taking step includes at least one of discarding infected meat, disinfecting equipment, isolation of infected animals, treating infected animals, or emergency slaughter of infected animals.

7. The method of claim 1, wherein the animal is one selected from the group consisting of ungulates, solipeds, birds, lagomorphs, farmed game, farmed game birds, and wild game.

8. The method of claim 1, wherein the consumer is a human or non-human animal.

9. The method of claim 1, wherein said step of assaying the sample comprises the step of screening for antibodies reactive to the lipogenic adenovirus in the sample.

10. The method of claim 9, wherein the antibodies are reactive to a lipogenic adenovirus protein selected from the group consisting of Ad-36 hexon protein and Ad-36 fiber coat protein.

11. The method of claim 9, wherein the antibodies in said screening step are reactive to one or more peptides encoded by the peptide sequences selected from the group consisting of SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, SEQ ID NO.: 15, SEQ ID NO.: 16, SEQ ID NO.: 17, and SEQ ID NO.: 18.

12. The method of claim 9, wherein said screening step is performed by using a method selected from the group consisting of serum neutralization assay and ELISA.

13. The method of claim 1, wherein said step of assaying the sample comprises the step of screening for lipogenic adenovirus nucleic acids.

14. The method of claim 13, wherein the nucleic acids are nucleic acids encoding a hexon protein.

15. The method of claim 14, wherein the hexon protein is encoded by a nucleic acid sequence of SEQ ID NO. 29.

16. The method of claim 13, wherein the nucleic acids are nucleic acids encoding the fiber coat protein.

17. The method of claim 16, wherein the nucleic acid sequence comprises SEQ ID NO.: 30.

18. The method of claim 13, wherein the nucleic acids in said screening step are detected using one or more nucleic acids selected from the group consisting of SEQ ID NO.: 19, SEQ ID NO.: 20, SEQ ID NO.:21, SEQ ID NO.:22, SEQ ID NO.:23, SEQ ID NO.:24, and SEQ ID NO.:25.

19. The method of claim 1, wherein the lipogenic adenovirus is one or more lipogenic adenoviruses selected from the group consisting of adenovirus type 5, adenovirus type 36, and adenovirus type 37.

20. The method of claim 1, wherein the sample is selected from the group consisting of a biological sample, body fluid, a tissue sample, an organ sample, feces, blood, saliva, and any combination thereof.

21. (canceled)

22. (canceled)

23. A method for evaluating the suitability of experimental animals or animal co-products for experimental use, said method comprising the steps of:
screening a sample from an animal designated for experimentation to determine whether the animal is infected with a lipogenic adenovirus; and
taking corrective action if the animals is positive for lipogenic adenovirus infection.

24. The method of claim 23, wherein the experimental animals are selected from the group consisting of rats, guinea pigs, rabbits, non-human primates, cats, dogs, horses, humans, birds, cows, sheep, goats, and chickens.

25. The method of claim 23, wherein the animal co-product is selected from the group consisting of biological sample, blood, semen, saliva, serum, cerebral fluid, urine, and plasma.

26. The method of claim 23, wherein if the animal is positive for lipogenic adenovirus infection, the animal is not suitable for experiments directed to fat metabolism, glucose metabolism, energy metabolism, cancer biology, or obesity.

27. The method of claim 23, further comprising conducting research on animals that are not infected and/or segregating data from experiments based on the presence or absence of the adenovirus.

28. The method of claim 23, further comprising not conducting research on animals infected with lipogenic adenovirus.

29. The method of claim 23, wherein said step of taking corrective action comprises vaccinating animals not infected with lipogenic adenovirus and/or treating animals infected with lipogenic adenovirus with an antiviral agent.

30. The method of claim 23, wherein said step of screening the sample comprises the step of screening for antibodies reactive to the lipogenic adenovirus in the sample.

31. The method of claim 30, wherein the antibodies are reactive to a viral protein selected from the group consisting of Ad-36 fiber coat protein and Ad-36 hexon protein.

32. The method of claim 30, wherein the antibodies in said screening step are reactive to one or more peptides encoded by the peptide sequences selected from the group consisting of SEQ ID NO.: 1, SEQ ID NO.: 2, SEQ ID NO.: 3, SEQ ID NO.: 4, SEQ ID NO.: 5, SEQ ID NO.: 6, SEQ ID NO.: 7, SEQ ID NO.: 8, SEQ ID NO.: 9, SEQ ID NO.: 10, SEQ ID NO.: 11, SEQ ID NO.: 12, SEQ ID NO.: 13, SEQ ID NO.: 14, SEQ ID NO.: 15, SEQ ID NO.: 16, SEQ ID NO.: 17, and SEQ ID NO.: 18.

33. The method of claim 30, wherein said screening step is performed by using a method selected from the group consisting of serum neutralization assay and ELISA.

34. The method of claim 23, wherein said step of assaying the sample comprises the step of screening for lipogenic adenovirus nucleic acids.

35. The method of claim 34, wherein the nucleic acids are nucleic acids encoding a hexon protein.

36. The method of claim 35, wherein the hexon protein is encoded by the nucleic acid of SEQ ID No. 29.

37. The method of claim 34, wherein the nucleic acids encode the fiber coat protein.

38. The method of claim 37, wherein the nucleic acid sequence comprises SEQ ID No.: 30.

39. The method of claim 34, wherein the nucleic acids in said screening step are detected using one or more nucleic acids selected from the group consisting of SEQ ID NO.: 19, SEQ ID NO.: 20, SEQ ID NO.:21, SEQ ID NO.:22, SEQ ID NO.:23, SEQ ID NO.:24, and SEQ ID NO.:25.

40. The method of claim 23, wherein the lipogenic adenovirus is one or more lipogenic adenoviruses selected from the group consisting of adenovirus type 5, adenovirus type 36, and adenovirus type 37.

41. The method of claim 23, wherein the sample is selected from the group consisting of a biological sample, body fluid, a tissue sample, an organ sample, feces, blood, saliva, and any combination thereof.

42. (canceled)

43. (canceled)
